(19) United States
(12) Patent Application Publication
(10) Pub. No.: US 2025/0075160 A1
(43) Pub. Date: Mar. 6, 2025

(54) Multi-chamber Cell Culture System and Method

(71) Applicant: POW Genetic Solutions, Inc., Emeryville, CA (US)

(72) Inventors: Shannon HALL, San Francisco, CA (US); Ouwei WANG, San Francisco, CA (US); Brian LEE, San Francisco, CA (US); Stephen HUBBARD, Oakland, CA (US)

(21) Appl. No.: 18/287,288

(22) PCT Filed: Jun. 28, 2023
(86) PCT No.: PCT/US2023/026512

§ 371 Date: Oct. 17, 2023

Related U.S. Application Data
(60) Provisional application No. 63/356,974, filed on Jun. 29, 2022.

(51) Int. Cl. C12M 1/00; C12M 1/26; C12M 1/34

(52) U.S. Cl. CPC C12M 23/58; C12M 29/00; C12M 33/14; C12M 41/36; C12M 41/44

(57) ABSTRACT

System and methods for continuous culture comprise performing a first continuous culture of cells in a chamber of a first bioreactor under culture conditions that produce cell growth; moving culture fluid comprising cells from the chamber of the first bioreactor into a chamber of a second bioreactor though one or more fluidic conduits; and performing, in the chamber of the second bioreactor, a second continuous culture of cells under culture conditions that produce at least one culture product. The process is performed under conditions wherein the rate of cell growth in the first continuous culture is greater than that of the second continuous culture, and the production of culture product in the first continuous culture is less than that of the second continuous culture.

### REFERENCE TO RELATED APPLICATIONS

This application claims the benefit of the priority date of U.S. provisional patent application 63/356,974, filed Jun. 29, 2022 (“Multi-chamber Cell Culture System and Method”), the contents of which are incorporated herein by reference in their entirety.

### STATEMENT AS TO FEDERALLY SPONSORED RESEARCH

This invention was made under a CRADA FP00011895 between Pow Genetic Solutions, Inc. and The Regents of the University of California Ernest Orlando Lawrence Berkeley National Laboratory operated for the United States Department of Energy. The Government has certain rights in this invention.

### BACKGROUND

The booming biomanufacturing and synthetic biology industries rely on bioreactors (also called fermenters) to bio-transform raw materials into value-added products. Biomanufacturing is a nascent sector that can be leveraged to produce a large share of the global economy's physical materials, not only with improved performance but also with better environmental footprint to combat climate change. Recent estimates place the total annual economic impact of synthetic biology at $2-4 trillion by 2040. Two thirds of this impact are outside of healthcare—in agriculture, consumer products, biomaterials, biochemicals, bioenergy, and many other areas. Among all potential applications of synthetic biology, the biomanufacturing of alternative proteins, biomaterials, biochemicals, and biofuels will account for the largest growth the next 10 to 20 years.

Further, the petrochemical industry has enabled the manufacturing of ubiquitous products that form the backbone of modern society—ranging from pharmaceuticals to everyday household goods. Petroleum-based processes, however, are unsustainable and contribute to global climate change. Biomanufacturing of renewable alternatives has largely not been able to compete on price, as the underlying production process, batch or fed-batch fermentation, has not changed in decades. Replacing the common batch and fed-batch bioreactor systems with a continuous-flow bioreactor would minimize equipment downtime, increase volumetric productivity, and reduce capital investment and operational costs. However, continuous-flow bioreactors are currently considered unreliable and are rarely used in biomanufacturing processes outside of pharmaceutical industries owing to the contamination and strain stability problems. Therefore, there is a need to develop reliable continuous-flow bioprocesses.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows results from a turbidostat in which temperature and pH are maintained constant over time and cells are maintained at a constant concentration/density (AU).

FIG. 2 shows an exemplary multi-chamber cell culture system comprising a first bioreactor (FIG. 2A) in fluid communication with a second bioreactor (FIG. 2B), optionally, in fluid communication with a cell-recycling system (FIG. 2C).

FIGS. 3A and 3B show an exemplary bioreactor.

FIG. 4 shows an exemplary computer system.

FIG. 5 shows an exemplary process for producing a culture product using a multi-chamber cell culture system.

FIG. 6 shows an exemplary hollow fiber cartridge for recycling cells into a second continuous cell culture. The cartridge 601 includes a feed port 607 connected to an effluent of the second bioreactor containing the second continuous culture, retentate port 609 that feeds cells back into the second continuous culture, hollow fibers 605 that form a semipermeable membrane and through which culture medium containing culture product can pass, but through which cells cannot pass, and permeate port 603 through which culture medium containing culture product exits the cartridge.

### SUMMARY

Disclosed herein are multi-chamber cell culture systems and methods for producing culture products. The multi-chamber cell culture systems comprise one or a plurality of first, and one or a plurality of second bioreactors in fluid communication with each other. Accordingly, a first bioreactor can feed a single second bioreactor; a first bioreactor can feed a plurality of second bioreactors; a plurality of first bioreactors can feed a single second bioreactor; or a plurality of first bioreactors can feed a plurality of second bioreactors.

In one embodiment, cells that produce a culture product of interest are grown in a chamber of one or more first bioreactors under conditions to maintain cell growth, but to produce little or none of the culture product (i.e., no more than 5% the amount of product produced in the second bioreactor). During cultivation, culture fluid comprising growing cells is directly transferred to chambers of one or more second bioreactors. Culture conditions in the second bioreactor are set to produce a culture product and to limit the growth rate of the cells therein. Limiting the growth rate of cells in the second culture container can inhibit the production and growth of mutant microorganisms that interfere with culture product production. In some embodiments, cells in the second bioreactor are not growing will have very slow growth rates, e.g., doubling times of at least one week. Culture product is removed from the second bioreactor, for example, by removing cell culture medium and isolating to sell product therefrom. In some embodiments, cells removed with a cell culture medium are recycled back into the second culture container.

In another embodiment, cells that can produce a product of interest are grown in a first bioreactor under conditions to maintain cell density. At steady-state, cells in such a culture will grow at a constant growth rate (e.g., log phase or exponential growth). In certain embodiments, the culture conditions are set to produce a maximum or near maximum growth rate. This can be accomplished, for example, by providing cell culture medium in which no nutrient is limiting for cell growth.

Growing cells from the first bioreactor are transferred through a fluidic conduit into each of one or more second bioreactors. Culture conditions in the second bioreactor(s) are maintained for production (preferably optimal production) of one or more desired culture products. This can include growing the transferred cells in the second bioreactor under conditions of no growth or very low growth. This can be accomplished with a culture medium that includes growth limiting amounts of a nutrient, such as a micronutrient.

Culture fluid from the second bioreactor is removed and the culture product is isolated from it. In some embodiments cells in the effluent are returned to the second bioreactor for continued production of the culture product.

Systems to perform these methods employ feedback mechanisms to maintain culture conditions and regulate the volume of the cell cultures. The feedback mechanisms involve sensors that measure one or more culture parameters of cell cultures in each bioreactor, computer modules to calculate culture parameters and determine if they are above or below a predetermined set points for these parameters, and effectors to adjust culture conditions toward the parameter set points. Such parameters include, for example, cell density, culture volume, temperature, pH, dissolved oxygen, and nutrient concentration. Effectors can include, for example, pumps to move liquids from reagent reservoirs into bioreactor vessels and from bioreactor vessels into other bioreactor vessels or to collection containers. Other factors can include, for example, temperature regulators to regulate temperature of cell cultures and air pumps to introduce oxygen into cell cultures. By use of such feedback mechanisms, various culture parameters can be maintained over time.

### DETAILED DESCRIPTION

### I. Continuous Culture

Methods and systems provided herein use continuous culture of cells to produce desired culture products. Any suitable culture product can be produced, such as a small molecule, a protein, a polynucleotide, and/or a cell.

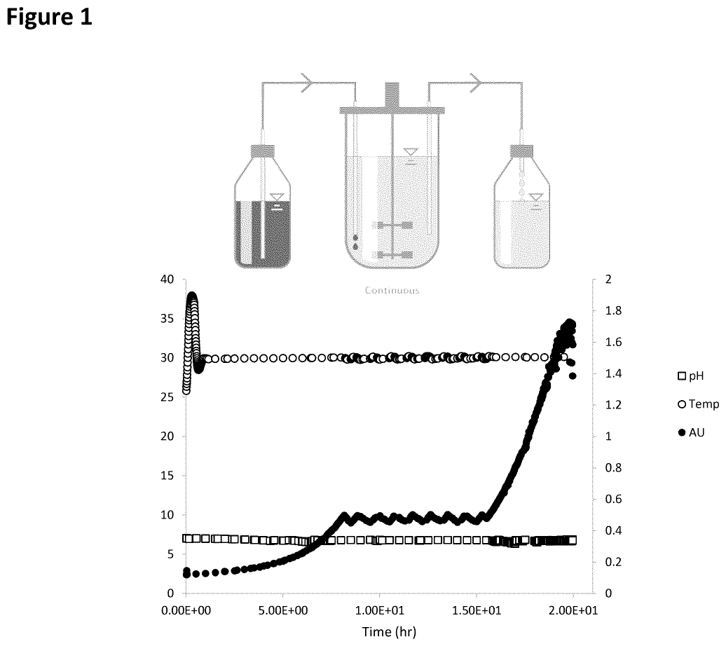

Two primary methods of culturing cells are batch culture and continuous culture. Batch culture is a closed system in which a cell culture, such as fermentation, is carried out with a fixed amount of nutrients. Continuous culture is an open system in which nutrients are continuously added to the culture. An example of continuous culture, a turbidostat, is shown in FIG. 1. Specifically, FIG. 1 shows maintenance of cells at a steady state of growth over time. Cell concentration is indicated by “AU”. A turbidostat maintains constant turbidity of the culture, which reflects cell density. Culture parameters are initially set and allowed to reach steady state. This may require, for example, 3 to 5 or more generations of cells. The turbidostat maintains cell density by removing culture medium containing cells from the bioreactor and replacing the volume with fresh medium. Accordingly, growth rate is a function of the amount of culture medium removed over time when the culture is at steady state. Batch culture is characterized by initial rapid growth of cells followed by a plateau after nutrients are used up which limits further growth. Continuous culture is typically characterized by a steady growth rate and/or a steady rate of culture output, as nutrients are continuously added to the system and cell density is maintained, for example, by removal of excess cells from the culture. Additional examples of continuous cultures include chemostat and perfusion culture.

Culture processes embrace any culturing of cells for producing a culture output (i.e., bioproduction to produce a bioproduct). These processes include, without limitation, processes referred to as “fermentation” and “cell culture.” “Fermentation” generally refers to enzymatic conversion of molecules into different molecular species (e.g., conversion of sugar into ethanol), typically by single-celled organisms. “Cell culture” generally refers to the culture of single cells, e.g., mammalian cell or insect cells, for the production of a product, such as a polypeptide or a value-added organic compound or the biomass. Any suitable cell can be used for culture (archaea, prokaryotes and/or eukaryotes), such as a fungal cell, for example yeast (e.g., Saccharomyces spp, Pichia spp, Komagataella spp, Kuyveromyces spp, Aspergillus spp, Rhodoporidium spp, Lipolytica spp, Aspergillus spp, Neurospora spp Trichoderma spp, Candida spp, or Penicillium), a bacterial cell, for example Escherichia coli, Bacillus spp, Costridia spp, Streptomyces spp, Pseudomonas spp, Ralstonia spp, Shewanella spp, an animal cell, for example arthropods (e.g., insects, shrimp, lobster, crayfish and crabs); chordates (e.g., fish, amphibians, reptiles, birds (e.g., chickens or turkeys); mammals (e.g., human or non-human such as bovine, lamb, goat, pig, horse, dog, cat, primate)), a cell line, for example CHO (Chinese Hamster Ovary cells), BHK21 (Baby Hamster Kidney), NS0, Sp2/0 Murine Cell lines, insect cells (e.g., SP9, Sf9, sf21, S2) tobacco BY-2 cells, Oryza, Sativa or algal cells).

The processes further embrace synthetic biology methods, in which cells are engineered to produce molecular outputs, for example, by expression within the cell of enzymes along a synthetic pathway.

### II. Multi-Chamber Cell Culture System

Multi-chamber cell culture systems of the present disclosure comprise one or more first bioreactors and one or more second bioreactors in fluidic communication with the first bioreactor. Accordingly, the system can comprise a first bioreactor in fluid communication with a single second bioreactor through a fluidic conduit, more, a central first bioreactor in fluid communication with a plurality of second bioreactors.

In certain embodiments, the multi-chamber cell culture system further comprises one or more reagent reservoirs fluidically connected to the first and/or one or more second bioreactors. The reagent reservoir can comprise any suitable reagent, such as a growth medium, a nutrient source, a carbon source, for example glucose, a buffer and/or one or more reagents for adjusting a parameter of the culture, for example the pH of the culture medium, and/or a gas, for example oxygen.

In certain embodiments, the multi-chamber cell culture system further comprises a reservoir fluidically connected to the second bioreactor and configured to receive a culture medium effluent, preferably comprising a culture product, from the second bioreactor. In certain embodiments, the culture medium effluent comprises one or more cells from the culture. In certain embodiments, culture medium effluent from the second bioreactor is passed through a filter, wherein the filter retains one or more cells in the culture medium effluent, i.e., retentate, while allowing to pass culture medium, i.e., filtrate, comprising one or more culture products. In certain embodiments, the filtrate comprises fewer cells than the culture medium effluent from the second bioreactor. In certain embodiments, the filtrate is devoid of cells. In certain embodiments, the filter is at least about any of 50, 60, 70, 80, 85, 90, 95, 99, 99.5, 99.9, or 100% effective at retaining cells from the culture medium effluent from the second bioreactor. In certain embodiments, the filtrate has no more than about any of 50, 40, 30, 20, 15, 10, 5, 1, 0.6, 0.1, or 0% of the cells from the cell culture medium effluent from the second bioreactor. In certain embodiments, the one or more cells retained on the filter, i.e., retentate, are returned to the second bioreactor, (i.e., a cell-recycling system).

In certain embodiments, the multi-chamber cell-culture system further comprises one or more feedback mechanisms and/or control systems to maintain culture conditions and regulate the volume of the cell cultures. The feedback mechanisms comprise one or more sensors that measure one or more process parameters of a bioreactor, computer modules to calculate process parameters and determine if they are above or below a predetermined set points for these parameters, and effectors to adjust process parameters toward the desired set points. In certain embodiments, the desired set points are entered by an operator using a programable interface. In certain embodiments, the sets points are developed using one or more computer programs and one or more tests to produce an optimal result. Any suitable sensor can be used to measure a process parameter, such as a culture parameter, for example cell density, culture volume, temperature, pH, dissolved oxygen, and/or nutrient concentration. Any suitable effector can be used, such as a liquid pump to move liquids from a reservoir into bioreactor vessels, from one bioreactor vessel into other bioreactor vessels, or to collection containers, a heat and/or cooling source to maintain the temperature of the bioreactor, a mixing unit (for example, an impeller, a pneumatic agitator, a stir bar and plate) to stir the bioreactor, and/or an air pump to introduce a gas into the culture, for example, oxygen or nitrogen. Additional examples of sensors and effects are disclosed herein.

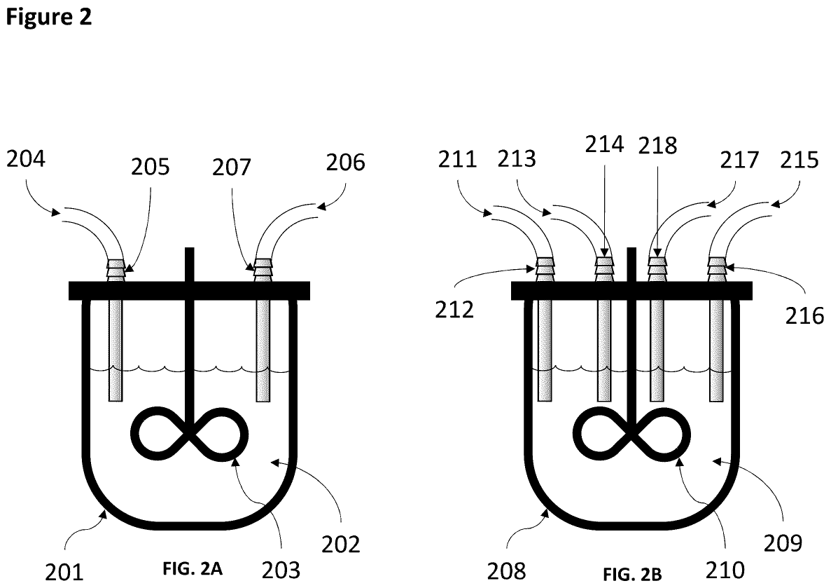

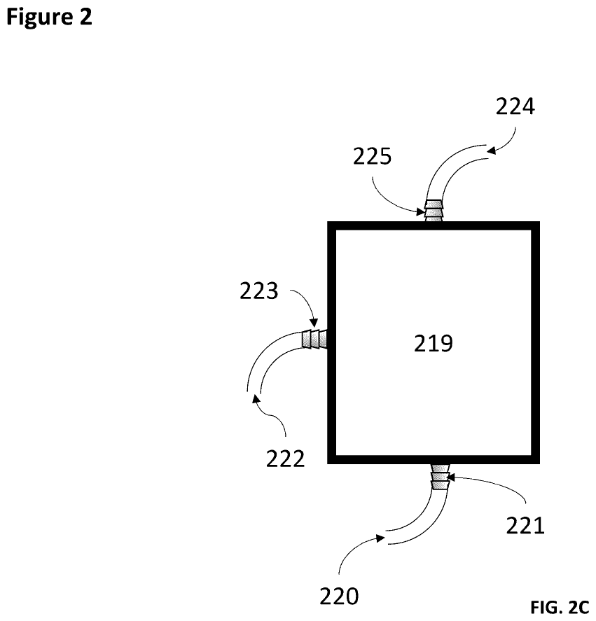

An exemplary illustration of a multi-chamber cell culture system is shown in FIG. 2. FIG. 2 illustrates a multi-chamber cell culture system comprising a first bioreactor (201, FIG. 2A) and a second bioreactor (208, FIG. 2B). The first bioreactor (FIG. 2A) is configured to comprise a first cell culture (202) capable of being mixed by a mixing unit (203). The first bioreactor further comprises a first inlet conduit (204) connected to a first inlet port (205) that is in fluid communication with a first reagent reservoir. The first bioreactor further comprises a first effluent conduit (206) (i.e., outlet) connected to a first effluent port (207), wherein the first effluent conduit (206) and port (207) are in fluid communication with both the first cell culture (203) and the second bioreactor (208). The second bioreactor (208) is configured to comprise a second cell culture (209) capable of being mixed by a mixing unit (210). The second bioreactor (208) further comprises a first inlet conduit (211) and first inlet port (212) fluidically connected to the first effluent conduit (206) and port (207) of the first bioreactor, configured to receive the first cell culture (203) from the first bioreactor (201). In certain embodiments, the first effluent conduit (206) of the first bioreactor (201) and the first inlet conduit (211) of the second bioreactor (208) are the same conduit. The second bioreactor (208) further comprises a second inlet conduit (213) connected to a second inlet port (214) that is in fluid communication with a second reagent reservoir. The second bioreactor (208) further comprises a first effluent conduit (215) connected to a first effluent port (216), wherein the first effluent conduit (215) and port (216) are in fluidic communication with the second cell culture (209).

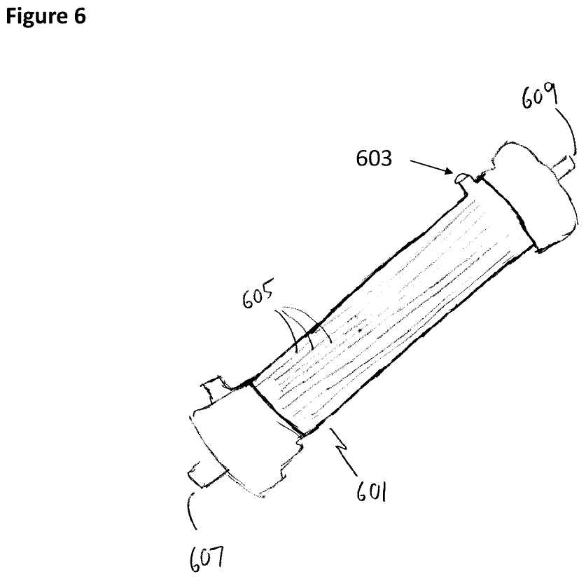

The second bioreactor can further comprise a cell-recycling system (219, FIG. 2C), wherein the cell-recycling system is configured to separate cells from their culture medium and (1) return (e.g., recycle) concentrated cell culture to the second bioreactor (208) through a third inlet conduit (217) connected to a third inlet port (218), or (2) transfer cells to a reservoir (not shown). The cell-recycling system comprises a first inlet conduit (220) connected to a first inlet port (221), wherein the first inlet conduit (220) and port (221) of the cell-recycling system are in fluid communication with the first effluent conduit (215) and port (216) and cell culture (209) of the second bioreactor. The cell-recycling system further comprises a first effluent conduit (222) connected to a first effluent port (223), both of which are in fluid communication with the third inlet conduit (217) and port (218) of the second bioreactor (208), wherein a concentrated cell culture is transferred from the cell-recycling system to the second bioreactor. The cell-recycling system further comprises a second effluent conduit (224) and second effluent port (225) configured to transfer the cell-free medium to a downstream process or reservoir. In certain embodiments, cell-recycling system comprises a filter, a membrane, or a hollow fiber cartridge operating as tangential flow or alternating tangential flow filtration system. Any suitable mechanism for separating cells from medium can be used, such as a centrifuge. An exemplary hollow fiber cartridge is depicted in FIG. 6.

The first and second bioreactor can comprise any suitable number of connected conduits, ports, each of which can be connected to a pump as necessary for the desired application. A port can be attached to the lid and/or the chamber of the bioreactor. A port can optionally comprise a dip tube. The dip tube can comprise any suitable length and diameter and either be in contact with the cell culture or not in contact with the cell culture. In either case, the port and/or dip tube can deliver and/or remove one or more liquid and/or gas components from a bioreactor. Any suitable material can be used for the ports and/or conduits. In preferred embodiments, the material is a biologically compatible material. In certain embodiments, the material is a chemically resistant material. The first and second bioreactor can comprise any suitable numbers of sensors and/or effectors as necessary for the desired application (as disclosed herein).

In certain embodiments, the multi-chamber cell culture system comprises a first bioreactor, one or more second bioreactors in fluid communication with the first bioreactor, one or more reagent reservoirs in fluid communication with the first bioreactor and, optionally, with the one or more second bioreactors, one or more pumps, and a control system. In certain embodiments, the first bioreactor comprises a first chamber and one or more first sensors providing at least a measure of cell density and a measure of volume of a cell culture in the first chamber. In certain embodiments, the one or more second bioreactors comprise a second chamber and one or more second sensors providing at least a measure of culture conditions and a measure of volume of a cell culture in the one or more second chambers. In certain embodiments, the one or more pumps are configured to (1) move liquid reagents from at least one reagent reservoir to the first chamber of the first bioreactor; (2) move cell culture fluid from the first chamber to the one or more second chambers; (3) move cell culture fluid out of the one or more second chambers; (4) move liquid reagents from at least one reagent reservoir to the one or more second chambers; and/or (5) move cell culture fluid from the second chamber of the second bioreactor to one or more reservoirs and/or downstream processes. In certain embodiments, the one or more pumps are configured for (1)-(3). In preferred embodiments, the one or more pumps are configured for (1)-(4). In more preferred embodiments, the one or more pumps are configured for (1)-(3), (5), and, optionally, (4). In certain embodiments, the control system is configured to (1) use measures from the one or more sensors to control cell density of a culture of cells in the first chamber of the first bioreactor; (2) move cell culture fluid from the first chamber of the first bioreactor to the one or more second chambers of the second bioreactor; (3) use measures from the one or more sensors to control volume and culture conditions of a culture of cells in the one or more second chambers of the second bioreactor; and/or (4) move cell culture fluid from the second chamber of the second bioreactor to the one or more reservoirs and/or downstream processes. In certain embodiments, the control system is configured for (1)-(3). In certain embodiments, the control system is configured for (1)-(4).

In certain embodiments, the multi-chamber cell culture system comprises a forward or reverse osmosis membrane.

### A. Bioreactor

Culture of cells is performed in a bioreactor. Bioreactors are systems that support and maintain a biologically active environment for a prescribed amount of time. Such systems comprise containers or chambers configured to contain culture medium, and elements to maintain or alter various culture parameters. A bioreactor can be configured to maximize any suitable parameter of the cell culture. In certain embodiments, a bioreactor can be configured to maintain an environment that supports optimal cell growth for the desired application. In certain embodiments, a bioreactor can be configured to maintain a desired cell density for the desired application. In further embodiments, a bioreactor can be configured to maintain an environment that supports optimal production for one or more desired culture products.

Batch and continuous bioreactor systems are commercially available from, for example, Sartorius (Goettingen, Germany), and ThermoFisher (Waltham, MA).

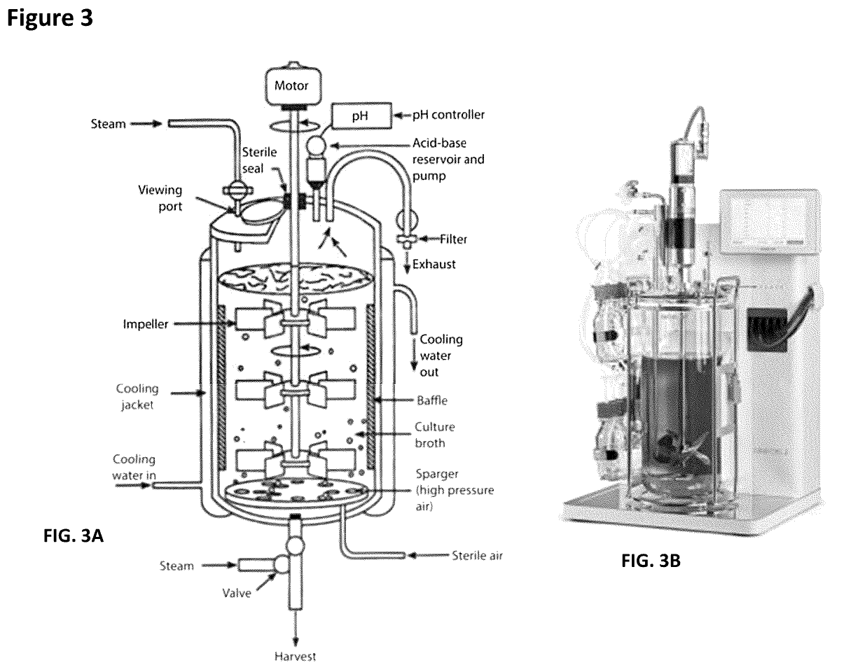

FIGS. 3A and 3B show exemplary bioreactors. FIG. 3A shows an exemplary schematic and componentry of a bioreactor. FIG. 3B shows a bioreactor attached to a computer with a user interface that indicates parameters of the culture than that accepts user instructions for controlling culture conditions. Reagent reservoirs, e.g., bottles, comprising nutrients of various sorts are attached to the culture through conduits, for example, a tube. Reservoirs may also accept excess culture medium from the culture and/or one or more culture products in a culture medium or culture fluid.

Bioreactors are systems configured to grow cultures of cells, typically by regulating one or a plurality of culture conditions and/or process parameters. Any suitable bioreactor configuration can be used, such as batch and/or continuous culture modes. In preferred embodiments, bioreactors are run in a continuous culture mode, for example: (1) turbidostat mode: dynamically adjusts the flow rate of nutrient delivery and periodically removing cells to keep the turbidity inside the vessel constant; (2) chemostat mode: cell growth is limited and controlled by a nutrient addition (for example, glucose, oxygen, glutamine) and depleted media with growth inhibitors is removed at approximately the same rate; and (3) perfusion mode: cells are either retained in the bioreactor or recycled back into the bioreactor, and fresh medium is provided and cell-free supernatant removed at the same rate.

### 1. Culture Chamber

Culture chambers of bioreactors generally have volumes between about 50 mL and about 50 L. This includes, for example, volumes between about 1 L and about 10 L. Industrial size fermenters can have sizes between about 50 L and about 1,000,000 L. In certain embodiments culture chambers can have volumes of between about 1,000 L and about 10,000 L. The multi-chamber cell culture chamber system can comprise any suitable culture chamber volume and any suitable combination of culture chambers volumes for each bioreactor. In certain embodiments, the culture chambers of the first and one or more second bioreactors comprise the same volume. In certain embodiments, the culture chambers of the first and one or more second bioreactors comprise a different volume. For example, the volume of the first culture chamber can be smaller than the volume of the second culture chamber. In certain embodiments, one or more of the culture chambers of the one or more second bioreactors comprise a different volume.

The multi-chamber cell culture system can comprise any suitable number of first bioreactors, such as at least any of 1, 2, 3, 4, 5, 6, 7, 8, 9, 10, 12, 14, 16, or 18 and/or not more than any of 2, 3, 4, 5, 6, 7, 8, 9, 10, 12, 14, 16, 18 or 20 first bioreactors, for example 1-20, preferably 1-10, more preferably 1-5, even more preferably 1-2. Typically a multi-chamber cell culture system comprise one first bioreactor. Additionally or alternatively, the multi-chamber cell culture system can comprise any suitable number of second bioreactors in fluidic communication with the one or more first bioreactors, such as at least any of 1, 2, 3, 4, 5, 6, 7, 8, 9, 10, 12, 14, 16, or 18 and/or not more than any of 2, 3, 4, 5, 6, 7, 8, 9, 10, 12, 14, 16, 18 or 20 second bioreactors, for example 1-20, preferably 1-10, more preferably 1-5, even more preferably 1-2. In preferred embodiments, the multi-chamber cell culture system comprises at least 1 first bioreactor and at least one second bioreactor. In even more preferred embodiments, the multi-chamber cell culture system comprises one first bioreactor and one or more second bioreactors.

In certain embodiments, a chamber comprises one or more baffles. A baffle can assist in stirring/agitation of the cell culture, which can assist, for example, in nutrient distribution, mixing of one or more reagents, and/or aeration.

Culture chambers include one or more ports into which liquids and/or gases can be introduced (“inputs”) into or from which liquids and/or gases can be removed from (“effluents”) the culture chamber. Each chamber can comprise any suitable number and/or combination of ports as necessary for the desired application. Such ports can communicate with fluid lines/conduits that communicate with other vessels, such as, reagent reservoirs, other culture vessels, collection reservoirs, and/or gas sources. Such fluid lines can comprise tubing, such as flexible tube. Any suitable tubing material can be used, for example, glass, metal, plastic, rubber, and/or a halogenated polymer. Preferred materials include biologically compatible materials and/or chemically resistant materials, for example, silicone, stainless steel, neoprene, polypropylene, pharmed, and/or Tygon. Movement of fluid through such tubing can be controlled with valves, such as pressure valves, one-way valves, two-way valves, three-way valves, shear valves, pinch valves, piezo valves, solenoid valves, and/or with pumps, such as peristaltic pumps, syringe pumps, piezo pumps, diaphragm pumps. Any suitable pump and/or valve combination can be used. Typically, a pump can be selected based on the minimum and maximum volume transfer needed, e.g., feed rate and/or media exchange rate, and therefore the flow rate requirement, which will depend on both the application and volume of the cell culture system, such as 100's of uL/min to 100's of L/min.

In certain embodiments, bioreactors and their chambers can comprise one or more feeds for the addition of nutrients and other molecules as well as one or more effluents to remove culture liquid from the bioreactor. In certain embodiments, the first bioreactor of the multi-chamber cell culture system comprises at least one feed for nutrient, and at least one effluent fluidically connected to the one or more second bioreactors. In certain embodiments, the second bioreactor of the multi-chamber cell culture system comprises at least one feed for cell culture from the first bioreactor, at least one feed for nutrient, and at least one effluent.

### B. Sensors and Effectors

Multi-chamber cell culture systems can further comprise one or a plurality of sensors for measuring various culture parameters, for example metabolic processes and process variables, and effectors for regulating these parameters. In preferred embodiments, the multi-chamber cell culture system comprises at least one sensor. In certain embodiments, the multi-chamber cell culture systems can comprise a sensor that (1) directly measures one or more metabolites (for example, acetate and ammonia, (2) detects changes in substrate (e.g., glucose), (3) or measure metabolism indirectly through cellular respiration by detecting changes in gas composition (e.g., oxygen and carbon dioxide). In certain embodiments, one or more culture parameters are measured and provide data to a feedback mechanism to direct feeding strategies and/or process conditions, among others. Any suitable number and combination of sensors can be used.

In certain embodiments, a sensor can be an “in-line” or “in situ” sensor, wherein the sensor shares a direct interface with the culture or a component of the bioreactor. Data from in-line sensors can be monitored and/or analyzed continuously or in a punctuated manner. The continuity of the measurement typically depends on the response time of the signal, the flow rate of the sampling procedures, and/or needs of the specific application. In certain embodiments, a sensor can be an “off-line” sensor, wherein a sample is collected, either manually or in an automated fashion, such as an autosampler, and analyzed in the laboratory, for example high-pressure liquid chromatography (HPLC), flow cytometry or microscopy analysis. In certain embodiment, the off-line measurement may be taken with a robotics or microfluidics system for analysis with instruments such as HPLC or gas chromatography (GC) in-line or off-line.

### a) Cell Density

In certain embodiments, one or more bioreactors in the multi-chamber cell culture system comprises a sensor that can measure the cell density, or proxy therefore, of a cell culture. For example, density of a culture is a function of optical density of the culture fluid. For example, one can measure optical density at OD₅₀₀ using a UV-Vis spectrophotometer. Other examples of spectroscopy for measuring cell density include mid-infrared (MIR), UV-VIS, fluorescence, Raman, and dielectric spectroscopy. Additionally, cell density is also function of capacitance of the cell culture. Therefore, one can measure capacitance of the culture fluid using a capacitance meter, such as a Hamilton probe or a Chloris probe. Furthermore, cell density can be measured using the acoustic resonance of the cell culture medium, where the specific gravity of the medium is used to determine the biomass. In certain embodiments, the sensor is capable of distinguishing the density component of living and/or viable cells from the density component of dead cells in the culture to provide an indication of the density of metabolically active cells in the culture.

Cell density of a culture can be affected by addition of fluid to a bioreactor vessel, to dilute the cell culture. The dilution rate for a continuous culture can be modeled using the formula D=F V⁻¹, wherein the dilution rate (D; [hr⁻¹]) is a function of the flow of medium into the bioreactor (F; [L hr⁻¹]) and the culture volume within the bioreactor (V; [L]). In certain embodiments, to reach a culture at steady state, the dilution rate (D) should be equal to the specific growth (p) of the cell culture, thereby keeping a constant biomass concentration in the bioreactor.

As discussed herein, pumps and valves can be used as effectors to move liquid into and out of a bioreactor. In certain embodiments, a multi-chamber cell culture system comprises a sensor capable of providing a measure of cell density to one or more feedback routines on a computer, wherein the computer actuates an effector to adjust the cell density to a desired set point. As discussed herein, pumps and valves can be used as effectors to move liquid into and out of a bioreactor as discussed above.

### b) Culture Volume

In certain embodiments, one or more bioreactors in the multi-chamber cell culture system can comprise a sensor that can measure cell volume or a proxy therefore. One such proxy for volume is mass. Accordingly, a bioreactor can be placed on a scale to measure the weight of the bioreactor. Additionally, or alternatively, a level sensor, such as a float valve or a capacitive fluid sensor, and/or an optical fluid sensor can be used. In certain embodiments, the optical fluid sensor is coupled with a level gauge.

Cell culture volume can be reduced by removing liquid culture from the bioreactor vessel, or increased by adding liquid to the bioreactor vessels. As discussed herein, pumps and valves can be used as effectors to remove liquid from a culture vessel. Alternatively, an overflow tube can function as a sensor and as a pump, for example a gravity pump. The overflow tube senses when the height of a culture exceeds the top of the overflow tube, and uses gravity to pull liquid through the tube and out of the vessel. In certain embodiments, both the volume and cell density (as described above) are maintained within a desired range. In certain embodiments, a multi-chamber cell culture system comprises a sensor capable of providing a measure of cell volume to one or more feedback routines on a computer, wherein the computer actuates an effector to adjust the cell volume to a desired set point.

c) pH

In certain embodiments, one or more bioreactors in the multi-chamber cell culture system can comprise a sensor that can measure pH of a cell culture. pH can be measured using any suitable pH meter, such as a pH electrode, for example an Ag/AgCl half-cell or an Ion Selective Field Effect Transistors (ISFET), or optical pH meter.

pH of a cell culture can be adjusted using a pumping system controlled by a feedback system connected to the pH sensor, wherein the pump system delivers a calculated volume of a suitable acid or base as necessary from a reservoir and/or titrates an amount of acid or base until the sensor reads the desired pH. In certain embodiments, a multi-chamber cell culture system comprises a sensor capable of providing a measure of pH to one or more feedback routines on a computer, wherein the computer actuates an effector to adjust the pH to a desired set point.

d) Dissolved Oxygen and/or Carbon Dioxide

In certain embodiments, one or more bioreactors in the multi-chamber cell culture system can comprise a sensor that can measure dissolved oxygen (“DO”) and/or dissolved carbon dioxide (“dCO₂”) in the culture fluid. Both dissolved oxygen and dissolved carbon dioxide can be measured with an optical sensor, a galvanic cell and/or a polarographic cell.

Dissolved oxygen can be controlled using an aeration system comprising a pump and/or valve in communication with one or more gas sources (for example a cannister or atmospheric air) that delivers air to an interior of one or more culture chambers. Dissolved carbon dioxide can be controlled by evacuating spent gas comprising CO₂ as the aeration system delivers new gases to the culture chamber. In certain embodiments, a multi-chamber cell culture system comprises a sensor capable of providing a measure of DO and/or dCO₂ to one or more feedback routines on a computer, wherein the computer actuates an effector to adjust the gas concentration in the bioreactor to a desired set point.

In certain embodiments, CO₂ can comprise a carbon source for the culture, therefore regulating dCO₂ in the culture aids the growth and/or production of one or more culture products. In certain embodiments, a culture may be maintained in a hypoxic or anaerobic atmosphere.

### e) Temperature

In certain embodiments, one or more bioreactors in the multi-chamber cell culture system can comprise a sensor that can measure temperature of the culture fluid. Temperature sensors, such as thermometers or thermistors, can be used for this purpose. In certain embodiments, the temperature sensor is placed in a thermowell, wherein the temperature sensor is capable of reading the culture temperature without being in direct contract with the culture itself.

Temperature of fluid cultures in culture chambers can be regulated using temperature regulators, such as, heating and/or cooling elements. Non-limiting examples of a heating element comprise a cartridge heater, a thin-film resistive heater, or a thermoelectric cooler (TEC). Non-limiting examples of cooling elements comprise a TEC or a cooling jacket. The temperature of one or more bioreactors in the multi-chamber cell culture system can be maintained at any suitable temperature, such as at least about any of 16, 18, 20, 22, 24, 26, 28, 30, 32, 34, 36, 38, 40, 42, 44, 46, 48, 50, 55, 60, 65, 70, 75, 80, or 85 and/or not more than 22, 24, 26, 28, 30, 30, 32, 34, 36, 38, 40, 42, 44, 46, 48, 50, 55, 60, 65, 70, 75, 80, 85 or 90° C., for example 20-90° C., preferably 24-50° C., more preferably 25-40° C. In certain embodiments, a multi-chamber cell culture system comprises a sensor capable of providing a measure of temperature to one or more feedback routines on a computer, wherein the computer actuates an effector to adjust the temperature in the bioreactor to a desired set point using a cooling and/or heating system.

### f) Glucose Concentration

In certain embodiments, one or more bioreactors in the multi-chamber cell culture system can comprise a sensor that can measure any suitable chemical component of the culture fluid. In preferred embodiments, the chemical component comprises a nutrient. In even more preferred embodiments, the nutrient comprises glucose, i.e., the concentration of glucose in the culture fluid. Glucose concentration can be measured with an electrochemical glucose sensor and/or spectroscopically. It can also be measured biochemically.

Glucose concentration of a cell culture can be adjusted using a pumping system controlled by a feedback system connected to the glucose sensor, wherein the pump system delivers a calculated volume of a fluid comprising glucose as necessary and/or titrates an amount of fluid comprising glucose until the sensor reads the desired glucose concentration. In certain embodiments, a multi-chamber cell culture system comprises a sensor capable of providing a measure of glucose concentration to one or more feedback routines on a computer, wherein the computer actuates an effector to adjust the glucose concentration in the bioreactor to a desired set point.

### g) Other Sensor and Effector Examples

As mentioned above, any suitable sensor can be incorporated to measure any suitable parameter (physical, chemical, and/or biological) as necessary for the desired application. Table 1 shows a non-limiting, exemplary list of parameters and sensors. Non-limiting examples of additional parameters include foam, viscosity, pressure, agitation rate, pyruvate concentration, lactate concentration, acetate concentration, cellular morphology, protein presence and/or concentration of a metabolite. Sensors to measure these parameters can be configured to measure conductance, capacitance, optical density, torque, or any other physical parameter. Further sensors can include spectroscopic, biochemical, optical, chromatography, cytometry, microscope, and/or electrochemical components to measure the desired parameter. One example includes liquid chromatography with UV detection.

TABLE 1
Parameter and Sensor Type examples

| Type | Parameter | Sensor Type |
|---|---|---|
| Physical | Temperature | Thermostat, thermistor |
| | Foam | Conductance |
| | Viscosity | Viscometer |
| | Pressure | Gauge |
| | Stirring | Torque |
| Chemical | Oxygen | Optical, electrochemical |
| | pH | Electrochemical, optical |
| | Lactate | Spectroscopic, biochemical |
| | Acetate | Spectroscopic, biochemical |
| | Glucose | Spectroscopic, biochemical |
| | Carbon Dioxide | Optical, electrochemical |
| Biological | Cell count (viable cell | Microscopy, spectroscopic |
| | density, total cell density, | |
| | viability, cell size, | |
| | aggregation) | |
| | Protein | Spectroscopy, chromatography |
| | Cellular morphology | Flow cytometry, spectroscopic |
| | Intermediate metabolites | Spectroscopy, chromatography |

In certain embodiments, a bioreactor can comprise a foam control mechanism, comprising a foam sensor and an effector for reducing foam, such as a tube and pump. In certain embodiments, a bioreactor can comprise a sparger and mass flow controller communicating with the chamber interior for input and/or displacement of one or more gases and/or mixtures thereof. In certain embodiments, a bioreactor can comprise a mixing unit operably connected to a motor for mixing/agitating the cell culture. Bioreactors also can contain stirring devices, such as paddles and ports for input of nutrients and other chemicals and for output of culture medium, which may include, cells.

### C. First Bioreactor

The first bioreactor can be any suitable bioreactor. In certain embodiments, the first bioreactor comprises a first chamber. The first chamber of the first bioreactor can be configured to hold any suitable volume, such as volumes between about 50 mL and 50 L, for example, volumes between about 1 L and about 10 L, volumes between about 5 L and 500 L, or volumes between 1,000 L and 10,000 L. In certain embodiments, the first chamber of the first bioreactor comprises a volume between about 250 mL and about 10 L. The first bioreactor can have a volume the same or less than the volume of the second bioreactor. For example, the first bioreactor can have a volume no more than any of 10%, 20%, 30%, 40%, 50%, 60%, 70%, 80%, or 90% that of the second bioreactor. This includes, for example, between 10% and any of 20%, 30%, 40%, 50%, 60%, 70%, 80%, or 90% that of the second bioreactor.

In certain embodiments, the first bioreactor further comprises one or more first sensors. The sensor can be any suitable sensor as disclosed herein. In preferred embodiments, the first bioreactor comprises a first sensor providing at least a measure of cell density and a first sensor providing at least a measure of volume of a cell culture in the first chamber. In certain embodiments, the first sensor providing at least a measure of cell density comprises an optical density or capacitance sensor. In certain embodiments, the first sensor providing at least a measure of volume of a cell culture comprises a scale measuring the mass of the bioreactor. In certain embodiments, the first bioreactor further comprises one or more first sensors that measure temperature, pH, and/or dissolved oxygen. In certain embodiments, the first bioreactor comprises a first sensor comprising a level sensor, such as an overflow tube), configured to move cell culture fluid from the first chamber when the height of the cell culture exceeds the top of the level sensor.

In certain embodiments, the first bioreactor comprises ports that communicate with reagent reservoirs and with each other through fluidic conduits. Any suitable number and configuration of ports on the first bioreactor can be used. In certain embodiments, the first bioreactor comprises a least about any of 1, 2, 3, 4, 5, 6, 7, 8, 9, 10, 12, 14, 16, 18, 20, 22, 24, 26, 28 30, 35, 40, or 45 and/or not more than about any of 2, 3, 4, 5, 6, 7, 8, 9, 10, 12, 14, 16, 18, 20, 22, 24, 26, 28 30, 35, 40, 45, or 50 ports, for example 1-50 ports, preferably 2-20 ports. In certain embodiments, a fluidic conduit is connected to the port, such as a tube. Any suitable number of reagent reservoirs can be connected to the first bioreactor.

In preferred embodiments, the first bioreactor is configured to be in fluid communication with the one or more second bioreactors.

In certain embodiments, the first chamber of the first bioreactor comprises a cell culture, and wherein the one or more reagent reservoirs in fluidic communication with the first chamber comprise growth medium in which one or more nutrients are present at a concentration that limits growth rate of the cells in the cell culture.

An exemplary first bioreactor is shown in FIG. 2A. The first bioreactor is configured to comprise a first cell culture (202) capable of being mixed by a mixing unit (203). The first bioreactor further comprises a first inlet conduit (204) connected to a first inlet port (205) that is in fluid communication with a first reagent reservoir. The first bioreactor further comprises a first effluent conduit (206) (i.e., outlet) connected to a first effluent port (207), wherein the first effluent conduit (206) and port (207) are in fluid communication with both the first cell culture (203) and the second bioreactor (208).

### D. Second Bioreactor

The second bioreactor can be any suitable bioreactor. In certain embodiments, the first bioreactor comprises a second chamber. The second chamber of the first bioreactor can be configured to hold any suitable volume, such as volumes between about 50 mL and 50 L, for example, volumes between about 1 L and about 10 L, volumes between about 5 L and 500 L, or volumes between 1,000 L and 10,000 L. In certain embodiments, the first chamber of the second bioreactor comprises a volume between about 250 mL and about 10 L.

In certain embodiments, the second bioreactor further comprises one or more second sensors. The sensor can be any suitable sensor as disclosed herein. In preferred embodiments, the second bioreactor comprises a second sensor providing at least a measure of volume of a cell culture in the second chamber. In certain embodiments, the second sensor providing at least a measure of volume of a cell culture comprises a scale measuring the mass of the bioreactor. In certain embodiments, the second bioreactor further comprises one or more second sensors that measure temperature, pH, and/or dissolved oxygen. In certain embodiments, the second bioreactor comprises a second sensor comprising a level sensor, such as an overflow tube, configured to move cell culture fluid from the second chamber when the height of the cell culture exceeds the top of the level sensor.

In certain embodiments, the second bioreactor comprises ports that communicate with reagent reservoirs and with each other through fluidic conduits. Any suitable number and configuration of ports on the second bioreactor can be used. In certain embodiments, the second bioreactor comprises a least about any of 1, 2, 3, 4, 5, 6, 7, 8, 9, 10, 12, 14, 16, 18, 20, 22, 24, 26, 28 30, 35, 40, or 45 and/or not more than about any of 2, 3, 4, 5, 6, 7, 8, 9, 10, 12, 14, 16, 18, 20, 22, 24, 26, 28 30, 35, 40, 45, or 50 ports, for example 1-50 ports, preferably 2-20 ports. In certain embodiments, a fluidic conduit is connected to the port, such as a tube. Any suitable number of reagent reservoirs can be connected to the second bioreactor.

Any suitable number of second bioreactors can be operably connected to the multi-chamber cell culture system. In certain embodiments, the multi-chamber cell culture system can comprise any suitable number of second bioreactors (i.e., a plurality of second bioreactors), such as at least any of 1, 2, 3, 4, 5, 6, 7, 8, 9, 10, 12, 14, 16, or 18 and/or not more than any of 2, 3, 4, 5, 6, 7, 8, 9, 10, 12, 14, 16, 18 or 20 second bioreactors, for example 1-20, preferably 1-10, more preferably 1-5, even more preferably 1-2. In preferred embodiments, one or more of the second bioreactors are configured to be in fluid communication with the first bioreactor. In more preferred embodiments, each of the one or more second bioreactors are configured to be in fluid communication with the first bioreactor.

The second bioreactor can comprise any suitable number of second chambers (i.e., a plurality of second chambers), such as at least any of 1, 2, 3, 4, 5, 6, 7, 8, 9, 10, 12, 14, 16, or 18 and/or not more than any of 2, 3, 4, 5, 6, 7, 8, 9, 10, 12, 14, 16, 18 or 20 second bioreactors, for example 1-20, preferably 1-10, more preferably 1-5, even more preferably 1-2.

In certain embodiments, the one or more second chambers of the second bioreactor are the same volume as the first chamber of the first bioreactor. In certain embodiments, the one or more second chambers of the second bioreactor comprise a different volume than the first chamber of the first bioreactor. In certain embodiments, The one or more second chambers of the second bioreactor are at least about any of 0.1, 0.2, 0.3, 0.4, 0.5, 0.6, 0.7, 0.8, 0.9, 1, 2, 3, 4, 5, 6, 7, 8, 9, 10, 20, or 50 and/or not more than about any of 0.2, 0.3, 0.4, 0.5, 0.6, 0.7, 0.8, 0.9, 1, 2, 3, 4, 5, 6, 7, 8, 9, 10, 20, 50, or 100× the volume of the first chamber of the first bioreactor, for example 0.1-100×, preferably 0.5-50×, more preferably 1-40×. In certain embodiments the second chamber of the second bioreactor has a greater volume than the volume of the first chamber of the first bioreactor. For example, the second chamber can have a volume between two times and 100 times that of the first chamber of the first bioreactor. In certain embodiments, each of the one or more second chambers of the second bioreactor are the same volume. In certain embodiments, one or more of the second chambers of the second bioreactor comprise a different volume than the remainder of the one or more second chambers of the second bioreactor(s).

In certain embodiments, the one or more second chambers of the second bioreactor comprise a cell culture, and wherein the one or more reagent reservoirs in fluidic communication with the second chamber comprise growth medium in which one or more nutrients are present at a concentration that limits growth rate of the cells in the cell culture. In certain embodiments, the culture media is formulated such that a nutrient is not limited in the first bioreactor, but one or more nutrients are present at a concentration that limits growth in the one or more second bioreactors.

An exemplary second bioreactor is shown in FIG. 2B. The second bioreactor (208) is configured to comprise a second cell culture (209) capable of being mixed by a mixing unit (210). The second bioreactor (208) further comprises a first inlet conduit (211) and first inlet port (212) fluidically connected to the first effluent conduit (206) and port (207) of the first bioreactor, configured to receive the first cell culture (203) from the first bioreactor (201). In certain embodiments, the first effluent conduit (206) of the first bioreactor (201) and the first inlet conduit (211) of the second bioreactor (208) are the same conduit. The second bioreactor (208) further comprises a second inlet conduit (213) connected to a second inlet port (214) that is in fluid communication with a second reagent reservoir. The second bioreactor (208) further comprises a second effluent conduit (215) connected to a second effluent port (216), wherein the second effluent conduit (215) and port (216) are in fluidic communication with the second cell culture (209).

In certain embodiments, the one or more second bioreactors are in fluidic communication with one or more collection vessels. In certain embodiments, the one or more collection vessels are configured to receive cell culture comprising both cells and cell medium. In certain embodiments, the one or more collection vessels are configured to receive cell-free culture medium. In certain embodiments, the one or more second bioreactors are in fluidic communication with a cell-recycling system (219, FIG. 2C), wherein the cell-recycling system is configured to separate cells from their culture medium and (1) return concentrated cell culture to the second bioreactor (208) through a third inlet conduit (217) connected to a third inlet port (218), or (2) transfer cells to a reservoir (not shown). An exemplary cell-recycling system comprises a first inlet conduit (220) connected to a first inlet pot (221), wherein the first inlet conduit (220) and port (221) of the cell-recycling system are in fluid communication with the first effluent conduit (215) and port (216) and cell culture (209) of the second bioreactor. The cell-recycling system further comprises a first effluent conduit (222) connected to a first effluent port (223), both of which are in fluid communication with the third inlet conduit (217) and port (218) of the second bioreactor (208), wherein a concentrated cell culture is transferred from the cell-recycling system to the second bioreactor. The cell-recycling system further comprises a second effluent conduit (224) and second effluent port (225) configured to transfer the cell-free medium to a downstream process or reservoir.

### E. Reagents, Fluidic Conduits

### 1. Reagent Reservoirs

The system comprises one or more reagent reservoirs. Each reagent reservoir contains a liquid reagent of some sort. This can include, for example, nutrients such as carbon source, a nitrogen source, an acid and or a base source. The reagent can comprise any suitable carbon source, such as a sugar (e.g., glucose, xylose, sucrose, glycerol, or acetate), molasses, malt extract, starch, dextrin, fruit pulp, CO or CO₂). The reagent can comprise any suitable nitrogen source, such as amino acids or polypeptide, urea, ammonium salt (e.g., ammonium sulphate, ammonium phosphate or ammonia), corn steep liquor, yeast extract, peptone, and soybean meal).

In certain embodiments, the reagent reservoir can comprise a reagent comprising one or more of: a metal (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), an inducer, a salt, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol, antifoaming agents (e.g., antifoam 204, antifoam A, antifoam C), amino acids (e.g., glutamate, leucine, and tryptophan), nucleic acid bases (e.g., adenine, cytosine, thymine, uracil, and guanine), complex nutrients (e.g., yeast extract, peptone, tryptone, and casamino acids), a macro-nutrient, a micro-nutrient, and a cell growth factor.

The reagent reservoir can comprise any suitable combination of reagents, such as a growth medium comprising a mixture of salts, vitamins, carbon sources, and nitrogen sources.

Reagent reservoirs are in fluid communication with the first and/or second bioreactor vessels through fluidic conduits, such as tubing. The conduits can engage ports in the reagent reservoirs and in the bioreactor vessels.

### 2. Pumps, Valves, and Filters

Multi-chamber cell culture systems can comprise one or a plurality of pumps to pump liquids from one container to another. This includes, for example, pumping liquids from reagent reservoirs to the first and/or second bioreactor vessels and between first and second bioreactor vessels. The pumps can be under the control of the control system.

In certain embodiments, the one or more pumps are configured to (1) move liquid reagents from at least one reagent reservoir to the first chamber of the first bioreactor; (2) move cell culture fluid from the first chamber to the one or more second chambers; (3) move cell culture fluid out of the one or more second chambers; (4) move liquid reagents from at least one reagent reservoir to the one or more second chambers. In certain embodiments, the one or more pumps are configured for (1), (2), and (3). In preferred embodiments, the one or more pumps are configured for (1)-(4).

Any suitable pump can be used, for example a peristaltic pump, a diaphragm pump, a piezo pump, a gravity pump, and/or a syringe pump. In one embodiment one or more pumps are peristaltic pumps. Peristaltic pumps can be positioned on fluidic conduits between vessels to induce liquid to move through the fluidic conduits.

Multi-chamber cell culture systems can further comprise one or more valves to either prevent liquid from flowing and/or to direct the flow of fluid. Any suitable number and types of valves can be used. For example, a multi-chamber cell culture systems can comprise a first pump and first valve in fluid communication with a first reagent reservoir and a chamber of a bioreactor. As nutrient is used by the cell culture, the control system can actuate the valve to open and the enable pump to transfer a value of nutrient to the cell culture. After transfer is complete, the control system can actuate the valve to close and disable the pump.

In certain embodiments, the pumps, valves, and/or chamber ports comprise a fitting for a tube. In certain embodiments, the fitting for the tube provides fluidic communication between two chambers, a chamber and a reagent reservoir, or an effluent conduit from the second chamber, or the second chamber and an effluent conduit.

Sterility can be important for the operation of a continuous bioreactor, including a multi-chamber cell culture system. In certain embodiments, a filter is fluidically connected to a conduit, such that the liquid flowing through the conduit passes through the filter removing any undesirable material. In certain embodiments, the filter removes contaminating biological material from a fluid preventing the contaminating biological material from entering a bioreactor. In certain embodiments, the filter comprises a 0.1-0.2 um pore size.

### F. Control System

In certain embodiments, the control system is configured to (1) use measures from the one or more sensors to control cell growth, e.g., cell density, of a culture of cells in the first chamber of the first bioreactor; (2) move cell culture fluid from the first chamber of the first bioreactor to the one or more second chambers of the second bioreactor; (3) use measures from the one or more sensors to control volume and culture conditions of a culture of cells in the one or more second chambers of the second bioreactor; and/or (4) move cell culture fluid from the second chamber of the second bioreactor to the one or more reservoirs and/or downstream processes. In certain embodiments, the control system is configured for (1)-(3). In certain embodiments, the control system is configured for (1)-(4). Thus, the control system functions as a feedback system. Information from one or more sensors is processed by a computer to determine the status of one or more culture parameters. Where the parameters have deviated from chosen setpoints, the computer transmits an instruction to an effector to adjust that parameter toward the setpoint.

In certain embodiments, the control system sets a dilution rate of liquid reagent being moved into the first and/or one or more second chambers to maintain concentration of one or more nutrients. In certain embodiments, the control system maintains a cell density for the cell culture at or near a desired value (determined through the process or by an operator). In certain embodiments, the control system is provided a measure of optical density of the cell culture and actuates one or more effectors to add reagent to the cell culture and/or to remove cell culture from the chamber of the bioreactor. In certain embodiments, the control system comprises a user-programmable module that computes a dilution rate as a function of error between target optical density and measured optical density.

In certain embodiments, the control system sets a volume of a cell culture in the first chamber of the first bioreactor and/or the one or more second chambers of the second bioreactor. In certain embodiments, the control system comprises a level sensor (e.g., an overflow tube) that moves cell culture fluid from a chamber when the height of the cell culture exceeds the top of the level sensor. In certain embodiments, the control system comprises a feedback routine that calculates the volume of a cell culture in the one or more second chambers based on a measure received from the one or more sensors, and, if the volume is above a set point, actuates a pump to move liquid cell culture from the one or more second chambers.

In certain embodiments, the one or more sensors provide measures of one or more culture parameters in the first and/or one or more second chambers to the control system. In certain embodiments, the multi-chamber cell culture system further comprises one or more effectors to affect changes in the one or more culture parameters. In certain embodiments, the control system comprises one or more feedback routines that calculate one or more culture parameters based on the measures, and actuates the one or more effectors to adjust the culture parameters toward target levels.

In certain embodiments, the multi-chamber cell culture system comprises one or more sensors to provide measures of one or more of pH, temperature, and dissolved O₂ of a cell culture in the first and/or second one or more chambers. In certain embodiments, the system further comprises one or more of: (1) one or more reagent reservoirs comprising an acid and a base in fluid communication with the first and/or one or more second reservoirs; (2) one or more temperature controllers to control temperature of a cell culture in the first and/or one or more second chambers; (3) one or more aerators for aerating a cell culture in the first chamber and/or one or more second chambers. In certain embodiments, the control system comprises one or more one of the feedback routines that calculate a culture condition comprising one or more of pH, temperature, dissolved O₂, and nutrient concentration based on a measure received from the one or more sensors, and actuates: one or more pumps to move acid or base from the reagent reservoir into the second chamber, to adjust pH to a target pH, one or more temperature controllers to adjust temperature to a target temperature; and/or one or more aerators to aerate the cell culture to a target dissolved oxygen level.

### 1. Computer

Feedback routines provided herein can be executed by programmable digital computer.

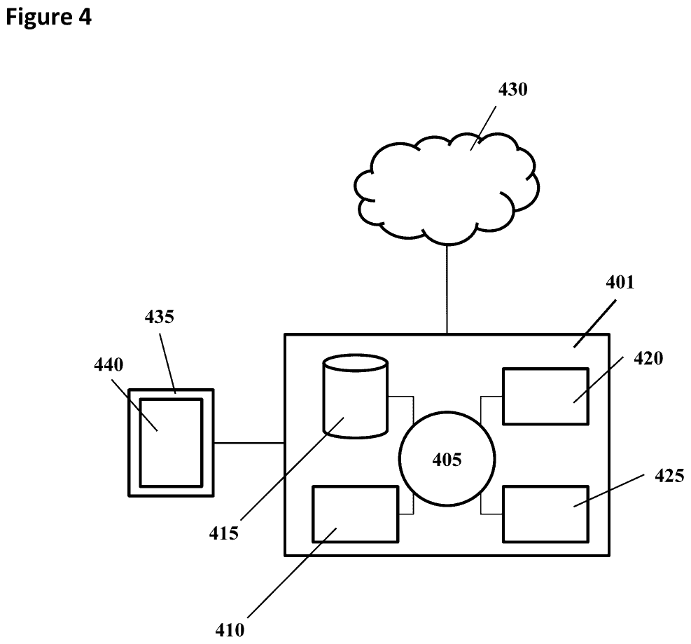

FIG. 4 shows an exemplary computer system. The computer system (401) includes a central processing unit (CPU, also “processor” and “computer processor” herein) (405), which can be a single core or multi core processor, or a plurality of processors for parallel processing. The computer system (401) also includes memory or memory location (410) (e.g., random-access memory, read-only memory, flash memory), electronic storage unit (415) (e.g., hard disk), communication interface (420) (e.g., network adapter) for communicating with one or more other systems, and peripheral devices (425), such as cache, other memory, data storage, bioreactors, and/or electronic display adapters. The computer readable memory (410), storage unit (415), interface (420) and peripheral devices (425) are in communication with the CPU (405) through a communication bus (solid lines), such as a motherboard. The storage unit 415 can be a data storage unit (or data repository) for storing data. The computer system (401) can be operatively coupled to a computer network (“network”) (430) with the aid of the communication interface (420). The network (430) can be the Internet, an internet and/or extranet, or an intranet and/or extranet that is in communication with the Internet. The network (430) in some cases is a telecommunication and/or data network. The network (430) can include one or more computer servers, which can enable distributed computing, such as cloud computing.

The CPU (405) can execute a sequence of machine-readable instructions, which can be embodied in a program or software (code). The instructions may be stored in a memory location, such as the computer readable memory (410). The instructions can be directed to the CPU (405), which can subsequently program or otherwise configure the CPU (405) to implement methods of the present disclosure.

The storage unit (415) can store files, such as drivers, libraries, and saved programs. The storage unit (415) can store user data, e.g., user preferences, log files, video or other images, and user programs. The computer system (401) in some cases can include one or more additional data storage units that are external to the computer system (401), such as located on a remote server that is in communication with the computer system (401) through an intranet or the Internet.

The computer system (401) can communicate with one or more remote computer systems through the network (430).

Methods as described herein can be implemented by way of machine (e.g., computer processor) executable code stored on an electronic storage location of the computer system (401), such as, for example, on the computer readable memory (410) or electronic storage unit (415). The machine executable or machine-readable code can be provided in the form of software. During use, the code can be executed by the processor (405). In some cases, the code can be retrieved from the storage unit (415) and stored on the memory 410 for ready access by the processor (405). In some situations, the electronic storage unit (415) can be precluded, and machine-executable instructions are stored on memory (410). The code can be used to communicate and issue instructions to electronic devices, e.g., circuit boards (440), modules, or subsystems, on the instrument.

The computer system (401) can communicate with one or more remote computer systems through the network (430).

Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks.

The computer system (401) can include or be in communication with an electronic display (435) that comprises a user interface (UI) (440) for providing, for example, input parameters for methods described herein. Examples of UIs include, without limitation, a graphical user interface (GUI) and web-based user interface.

In certain embodiments, the control system comprises a computer comprising a processor and memory comprising executable code which, when executed by the processor, performs one or more feedback routines to control a parameter of the bioreactor. In certain embodiments, the parameter of the bioreactor comprises the cell density in the first chamber, control volume of a cell culture in the first chamber and/or one or more second chambers and control the culture condition in the one or more second chambers control cell density. In certain embodiments, one of the feedback routines calculate the cell density of a cell culture in the first chamber based on a measure received from the one or more sensors, and actuates a pump to move liquid reagent from a reagent reservoir to the first chamber to adjust the cell density to a set point. In certain embodiments, one of the feedback routines calculates the volume of a cell culture in the first chamber based on a measure received from the one or more sensors, and, if the volume is above a set point, actuates a pump to move liquid cell culture from the first chamber to the one or more second chambers.

In another embodiment a feedback routine measures a parameter, such as cell density, in the first bioreactor and regulates the addition of culture medium or removal of cell culture to maintain cells at a growth phase; and measures one or more parameters related to culture conditions in the second bioreactor and regulates addition of medium and or nutrients, and removal of cell culture, to maintain cells in production phase.

### 2. Machine Learning

In certain embodiments, one or more data types from one or more parameters of a multi-chamber culture system may be stored and used to identify improvements in a process and/or culture parameter. In certain embodiments, provided is a network comprising a plurality of separate multi-chamber cell culture systems, wherein each of the systems send information regarding one or more aspects of one or more processes at the system to a central processing unit. In certain embodiments, one or more systems of the plurality of systems are spatially separated. The central processing unit can process the information and send output to one or more of the multi-chamber cell culture systems, such as output that causes a change in the one or more of the multi-chamber cell culture systems. In certain embodiments, the network comprises at least 2, 3, 4, 5, 6, 7, 8, 10, 12, 15, 20, 25, 30, 40, 50, 70, 100, 200, or 500 multi-chamber cell culture systems and/or not more than 3, 4, 5, 6, 7, 8, 10, 12, 15, 20, 25, 30, 40, 50, 70, 100, 200, 500, or 1000 multi-chamber cell culture systems. The central processing unit may be a single unit or a plurality of units, and can be, e.g., distributed, such as a cloud-based system. The processing unit can be configured to learn from information provided by the various systems and adjust conditions at one or more systems based, at least in part, on the learning, for example, by using a machine learning algorithm. Any suitable multi-chamber cell culture systems may be networked.

In some variations, an input to a feedback mechanism may be derived using methods involving implementing machine learning techniques including linear and non-linear models, e.g., processes such as CART—classification and regression trees), artificial neural networks such as back propagation networks, discriminant analyses (e.g., Bayesian classifier or Fischer analysis), logistic classifiers, and support vector classifiers (e.g., support vector machines) using the data types from the multi-chamber culture system.

Machine learning algorithms for identifying optimal culture conditions can take advantage of deep learning techniques. Deep learning techniques make use of multiple layers in the learning process.

One deep learning technique is reinforcement learning. Reinforcement learning is an aspect of machine learning where an agent learns to behave in an environment by performing certain actions in observing the rewards/results which it gets from those actions. An agent takes an action (aₜ) on its environment. This produces information about the environment state (Sₜ) and a reward (Rₜ) indicating whether the result is better than the previous result. The agent works on the hypothesis of reward maximization.

One version of reinforcement learning is referred to as direct search. “Direct search” refers to sequential examination of trial solutions involving comparison of each trial solution with the “best” obtained up to that time, together with a strategy for determining (as a function of earlier results) what the next trial solution will be.

Q-learning is a model-free reinforcement learning algorithm to learn the value of an action in a particular state.

Artificial neural networks use collections of interconnected nodes. Neural networks are comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network. comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network.

### III. Method of Producing a Product

The methods provided herein can use any suitable system, for example a multi-chamber cell culture system as described above.

Provided herein are methods of making a product in a multi-chamber cell culture system. The methods involve culturing cells in the first bioreactor under conditions to maintain cell growth, e.g. by maintaining a constant cell density. Under such constant cell density conditions, when cells reach a steady state, they are growing at a constant growth rate. In certain embodiments, cells are grown with a cell culture medium in which no nutrients are growth limiting and other culture conditions, such as, temperature, pH and dissolved oxygen are set to provide a maximum or near maximum growth rate.

In certain embodiments, the cells in the first culture chamber are then automatically moved into a second bioreactor chamber (or into each of a plurality of second bioreactor chambers) where culture conditions are set to optimize the production of a desired culture product.

In certain embodiments, culture fluid is removed from the second culture chamber and culture product is isolated from the fluid. In certain embodiments, cells in the removed culture fluid are returned to the second culture chamber to continue the production of product.

In certain embodiments, provided herein are methods for (1) performing a first continuous culture of cells in a first chamber of a first bioreactor; (2) during the first continuous culture, moving culture fluid comprise cells from the first chamber of the first bioreactor into a second chamber of at least one second bioreactor; (3) performing a second continuous culture of cells moved from the first bioreactor in the second chamber of the one or more second bioreactors to product a culture product; and (4) collecting the at least one culture product from the second chamber of the one or more second bioreactors. In preferred embodiments, the first bioreactor is operated under culture conditions that maintain cell growth by maintaining a constant cell density (e.g., turbidostat). In certain embodiments, the one or more second bioreactors are operated under constant culture conditions that favor production of a culture product. In certain embodiments, the constant culture conditions in the one or more second bioreactors are optimized for the formation of the culture product (e.g., chemostat). Any suitable culture conditions can be used depending on the cell and the application. In certain embodiments, the method further comprises maintaining a constant volume of the culture of cells in the chamber of the first bioreactor (as disclosed herein). In certain embodiments, a constant volume of cell culture in the first bioreactor is maintained by moving culture fluid from the first chamber of the first bioreactor, using for example a pump and/or valve, to the second chamber of the one or more second bioreactors when the volume of the first bioreactor exceeds the target volume. In certain embodiments, the cell density of the first and/or one or more second bioreactors is maintained. Any suitable sensor for cell density can be used (as disclosed herein). In certain embodiments, the sensor comprise optical density and the method comprises measuring the optical density of the cell culture and diluting the cell culture by adding fluid, for example nutrient liquid, and/or removing a portion of the cell culture fluid to maintain a target optical density. In certain embodiments, the culture conditions for the one or more second bioreactors are optimized for production of one or more culture products. In certain embodiments, the method comprises adding one or more inducer molecules to the culture of the one or more second bioreactors to induce activity of a biochemical pathway that produces a culture output (i.e., culture product). In preferred embodiments, the inducer molecule is not added to the culture of the first bioreactor. In certain embodiments, the growth rate of the culture in the second bioreactor is limited by nutrient maintained a doubling rate at least any of 2, 3, 5, 10, 15, 20, 50, 100× slower than the first bioreactor, preferably 10-100× slower. In certain embodiments, the method further comprises collecting/recovering the one or more culture products from the culture fluid.

### A. Turbidostat and Chemostat

Two methods of operating a bioreactor in continuous culture include turbidostat and chemostat. In both cases nutrients and other chemicals are added to the cell culture at a rate referred to as the dilution rate. The dilution rate is determined by feedback from sensors in the culture container to maintain a desired parameter.

A turbidostat (FIG. 1) is a continuous bioreactor in which cell density is kept constant. Additionally or alternatively, the culture volume of the turbidostat can be kept constant. Cell density or biomass is determined by a proxy measure such as turbidity or capacitance. Once cells reach a steady state, if cell density is maintained, the growth rate will be constant. In this system nutrient can be maintained in excess to achieve a maximum or desired growth rate. The maximal growth rates change depending on the medium formulation and process conditions. In certain embodiments, while culturing a cell culture under a turbidostat mode, the growth rate of the culture is maximized and/or set to a desired rate and, as a result, bioproduction of the culture product in limited. In certain embodiments, no culture product is generated while culturing a cell culture under a turbidostat mode. In certain embodiments, the turbidostat is coupled to a volume sensor to maintain both constant turbidity and volume by removing culture from the turbidostat once volume exceeds a set point.

A chemostat is a type of continuous bioreactor that maintains a steady-state of culture conditions. Culture conditions are maintained by continuously adding medium to the culture in order to maintain concentration of nutrients and other chemicals, while also removing culture liquid to keep the culture volume constant. The growth rate can be adjusted physically by changing the rate of addition of chemicals to the chemostat, and by altering rate of removal of culture liquid. The culture conditions can be set to favor or to optimize the production of a culture product. Such conditions typically divert cell machinery away from cell growth and toward the reduction of the culture product.

In certain embodiments, the method comprises operating a first bioreactor in a turbidostat mode and one or more second bioreactors in a chemostat mode.

### B. Culture Conditions

In either turbidostat mode or chemostat mode, a number of different culture conditions can be regulated.

### 1. Cell Density

Cell density can be maintained as follows. A setpoint of cell density is chosen. A measure of cell density is determined from sensors in the bioreactor. For example, a spectrometer can measure turbidity of the cell culture. If cell density is above the setpoint, culture medium is added to the culture chamber. If cell density falls below the setpoint, addition of culture medium is stopped until the growth of cells increases the cell density above the setpoint. In certain embodiments, the OD of the first bioreactor is at least about any of 0.5, 0.6, 0.7, 0.9, 1, 2, 3, 4, 5, 6, 7, 8, 9, 10, 12, 14, 16, 18, 20, 25, 30, 35, 40, 45, 50, 75, 100, 125, 150, 175, or 200 and/or no more than about any of 0.6, 0.7, 0.9, 1, 2, 3, 4, 5, 6, 7, 8, 9, 10, 12, 14, 16, 18, 20, 25, 30, 35, 40, 45, or 50, for example 1-50, 1-20, 1-10, 50-400, 50-300 or 100-200. In certain embodiments, the OD of the second bioreactor is at least about any of 0.5, 0.6, 0.7, 0.9, 1, 2, 3, 4, 5, 6, 7, 8, 9, 10, 12, 14, 16, 18, 20, 25, 30, 35, 40, 45, 50, 60, 70, 80, 90, 100, 150, 200, or 250 and/or no more than about any of 0.6, 0.7, 0.9, 1, 2, 3, 4, 5, 6, 7, 8, 9, 10, 12, 14, 16, 18, 20, 25, 30, 35, 40, 45, 50, 60, 70, 80, 90, 100, 150, 200, 250, or 300, for example 1-300, preferably 50-300, more preferably 50-200.

Cell density can be affected by addition of fluid to a bioreactor vessel, to dilute the cell culture. The dilution rate for a continuous culture can be modeled using the formula D=F V⁻¹, wherein the dilution rate (D; [hr⁻¹]) is a function of the flow of medium into the bioreactor (F; [L hr⁻¹]) and the culture volume within the bioreactor (V; [L]). In certain embodiments, to reach a culture at steady state, the dilution rate (D) should be equal to the specific growth (p) of the cell culture, thereby keeping a constant biomass concentration in the bioreactor. In certain embodiments, the method comprises a control system comprising one or more sensors and effectors that actively maintain the cell density of one or more bioreactors. In certain embodiments, the cell density for a bioreactor is inputted into a user-programmable module, and the control system actively maintains the cell density of the one or more bioreactors. In certain embodiments, maintaining the cell density comprises adding additional fluid, e.g., nutrient liquid, and/or removing culture fluid. In preferred embodiments, maintaining the cell density comprises both adding additional fluid and removing cell culture. As discussed herein, pumps can be used as effectors to move liquid into and out of a bioreactor.

### 2. Culture Volume

The volume of culture can be maintained in either or both of the turbidostat mode and the chemostat mode.

Mass of the bioreactor is a proxy for volume of the cell culture, as mass will increase or decrease with increases or decreases in volume. The system can decrease the volume of the cell culture when it gets too large by removing cell culture from the bioreactor. This can be done either with pumps or with a mechanical device such as a top off tube.

In certain embodiments, the method comprises a control system comprising one or more sensors and effectors that actively maintain the culture volume of one or more bioreactors. In certain embodiments, the culture volume for a bioreactor is inputted into a user-programmable module, and the control system actively maintains the culture volume of the one or more bioreactors. In certain embodiments, maintaining the culture volume comprises adding additional fluid, e.g., nutrient liquid, and/or removing culture fluid. In certain embodiments, maintaining the culture volume comprises both adding additional fluid and removing cell culture. As discussed herein, pumps can be used as effectors to move liquid into and out of a bioreactor.

### 3. Temperature

Temperature can affect both the growth rate of cells in a culture and their production of a product. Accordingly, temperature regulation is a culture condition that can be regulated in the methods herein using any suitable control system as described above. The temperature of one or more bioreactors in the multi-chamber cell culture system can be maintained at any suitable temperature, such as at least about any of 16, 18, 20, 22, 24, 26, 28, 30, 30, 32, 34, 36, 38, 40, 42, 44, 46, 48, 50, 55, 60, 65, 70, 75, 80, or 85 and/or not more than 22, 24, 26, 28, 30, 30, 32, 34, 36, 38, 40, 42, 44, 46, 48, 50, 55, 60, 65, 70, 75, 80, 85 or 90° C., for example 15-90° C., preferably 20-50° C., more preferably 25-40° C.

4. pH

The pH indicates the acidity or alkalinity of a culture. It can be regulated by adding a solution comprising an acid or base to the culture. pH of a cell culture can be adjusted using a pumping system controlled by a feedback system connected to a pH sensor, wherein the pump system delivers a calculated volume of a suitable acid or base as necessary and/or titrates an amount of acid or base until the sensor reads the desired pH.

### 5. Dissolved O₂

Dissolved oxygen is necessary for the growth of aerobic cells and microorganisms. It can be regulated using an aeration system to introduce air into the cell cultures. In certain embodiments, the method comprises controlled using an aeration system comprising a pump and/or valve in communication with one or more gas sources (for example a cannister or atmospheric air) that delivers air to an interior of one or more culture chambers. Dissolved carbon dioxide can be controlled by evacuating spent gas comprising CO₂ as the aeration system delivers new gases to the culture chamber.

In certain embodiments, the method comprises maintaining a desired gas concentration for one or more gases. In certain embodiments, the gas concentration in a cell culture is measured using a suitable sensor, and a control system comprising a feedback mechanism actuates one or more aeration systems comprising one or more gas pumps and/or valves in communication with one or more gas sources (for example a cannister or atmospheric air), wherein the pumping system delivers a calculated volume of a gas to an interior of one or more culture chambers. In certain embodiments, one or more gases (for example CO₂) is evacuated as the aeration system delivers new gases to the culture chamber.

### 6. Nutrient Concentration

The concentration of nutrients can be critical for growth rates. To maintain maximum growth rates the concentration of nutrients in a cell culture should be set so as to not be limiting for growth rate.

However, for the production of product, one may wish to set culture conditions to prefer the production of the product. In such circumstances, one may wish to decrease the concentration of one or more nutrients. For example, cells grown in turbidostat mode typically are growing at a high growth rate and require high levels of a nutrient. However, when they are moved to a bioreactor operating in chemostat mode, it may be necessary to decrease concentration of one or more nutrients, for example micronutrients, such that cellular physiology becomes directed to the production of a culture product. In certain embodiments, the growth rate of the culture in the second bioreactor is maintained under growth limiting (e.g., no growth or very slow growth) conditions. This can be, for example, a doubling rate of less than any of 24 hours, 48 hours, 72 hours, 96 hours, 120 hours, five days, six days, seven days, two weeks, or one month. In other embodiments the culture is maintained with a doubling rate of at least about any one of 72, 48, 24, 12, 6, or 3 hours. In certain embodiments, the growth rate of the culture in the second bioreactor is maintained with a doubling rate at least any of 2, 3, 5, 10, 20, 50, or 100× slower than that that of the first bioreactor.

In certain embodiments, the method comprises maintaining a desired nutrient concentration for one or more nutrients. In certain embodiments, the nutrient concentration in a cell culture is measured using a suitable sensor, and a control system comprising a feedback mechanism actuates one or more pumping systems in fluidic communication with one or more nutrient reservoirs, wherein the pumping system delivers a calculated volume of a nutrient liquid as necessary and/or titrates an amount of fluid comprising the sensor reads the desired nutrient concentration.

### C. Culture Products

A culture output can be any measurable characteristic of a culture to be optimized. Culture outputs include, for example, cell biomass, molecular products, and proxies of product production or cell health based on cell physiology. In certain embodiments, the method further comprises collecting/recovering the one or more culture products.

In certain embodiments, the culture output is the biomass of the cells themselves. In this case, the culture output to be optimized can be cell growth rate. Cell growth rate can be measured as a function of change in turbidity over time.

In certain embodiments, the culture output is a chemical product produced by the cells. (“culture product”). The culture product can be a molecular entity that is the product of fermentation or gene expression and which, typically, is a product to be harvested from the culture and commercialized. Culture products contemplated herein include polypeptides, e.g., proteins, enzymes, antibodies (e.g., monoclonal antibodies), vaccines, extracellular vesicles, and recombinant pharmaceutical proteins (e.g., hormones, growth factors, enzymes, and cytokines). Culture products also include organic molecules that are the product of synthetic pathways in the cell, e.g., mediated by enzymes. Such products include, for example, industrial chemicals. These include, without limitation, flavorings (e.g., vanillin); flagrances (e.g., aldehydes, coumarins, indoles), amino acids, organic acids (e.g., citric, lactic and acetic acids); alcohols (e.g., ethanol, isopropanol, ketones such as acetone); fatty acids (e.g., palmitic and oleic acid). The product output can be measured directly as concentration or as a function of amount, for example, as volumetric production rate, product titer, product yield, or specific production rate.

In certain embodiments, the culture output is a proxy of output production based on, e.g., cell physiology (“culture proxy”). Culture proxies are measurable parameters that indicate health and/or physiology of a culture of cells. These include, without limitation, specific CO₂ generation rate, specific O₂ consumption rate, organic acid profile, metabolite profile, side product profile, production economics (the cost to produce 1 kg of product under particular cell culture conditions). Specific rates can be defined as the rate of change of a compound per cell per hour, or volumetric production rate as the rate of change of a compound per liter of fermentation media per hour.

### D. Cell Growth in the First Bioreactor

In the first bioreactor, cells are grown under culture conditions adapted for continuous growth of cells and minimal or no production of a culture product.

The continuous growth rate can include a constant growth rate over the period in which cells are transferred to the second culture container. Growth rate can be measured as a function of doubling time. The growth rate can be a maximum growth rate. However, less than maximum growth rates may be desirable. Accordingly, the growth rate can comprise rates at least any of 1%, 2%, 4%, 8%, 16%, 32%, 50%, 60%, 70% 80% or 90% of the maximum growth rate, e.g., between 10% to 100% of the maximum growth rate.

In certain embodiments, the cells can be grown at constant density, typically under conditions optimized for high or desired growth rate. This will include, for example, use of cell culture medium in which no nutrient is at a growth limiting concentration.

In other embodiments, the culture medium comprises one or more nutrients, such as micronutrients, that are present in amounts to allow cell growth in the first bioreactor, but that will not support cell growth after transfer to the one or more second production bioreactors. For example, the nutrients may be present in the first bioreactor in amounts such that after transfer, any remaining nutrient is quickly consumed and the cells reach a steady state based on the amount of nutrient feeding the second bioreactor.

In certain embodiments, the methods provided herein comprise maintaining a constant growth rate in the cell culture. Any suitable method for maintain a constant growth rate can be used, for example limiting one or more reagents/substrates/nutrients and/or adjusting the temperature. In certain embodiments, the growth rate is at least any of 10, 20, 30, 40, 50, 60, 70, 80, 90, 95, or 100% of the maximum cell growth rate. In certain embodiments, wherein one or more reagents/substrates/nutrients are limiting, the growth rate is no more than any of 50, 60, 70, 80, 90, 95, or 100% of the maximum cell growth rate as compared to a culture not lacking the one or more reagents/substrates/nutrients. In certain embodiments, cell growth rate is maintained without nutrient limitation. In certain embodiments, cell growth rate is not limited or controlled.

Production of culture product in the first bioreactor can be less than the production of the culture product in the second bioreactor. For example, amount of product produced in the first bioreactor per unit volume can less than any of 50%, 40%, 30%, 20%, 10%, 5%, 3%, or 1% that of the second bioreactor. By “minimal” production of a culture product is meant less than commercially viable, e.g., the cost to produce the product is no more than two times the value of the product produced. This amount could be, e.g., 5% the amount when cell are cultured under product optimized conditions, or less than 0.05 moles per liter per hour. Culture product production can be measured in gms product per gm dry cell weight per hour. Production of a culture product can be suppressed by appropriately setting culture conditions. For example, if the product is produced via an induction process, the culture is not exposed to the inducer. Furthermore, by maintaining little to no production, the provided methods contribute to strain stability by reducing the metabolic burden on the primary strain, thereby decreasing the probability that mutant strains will develop.

In certain embodiments, culture fluid from the first bioreactor comprising the cells is moved into the second bioreactor periodically. This can be, for example, when the volume of the cell culture exceeds the setpoint. In that case, liquid removed from the culture chamber will be offset by culture medium added to that chamber.

### E. Cell Growth in the Second Bioreactor

In the second bioreactor or bioreactors, cells can be grown under culture conditions that produce a desired culture product. Such conditions can favor or be optimized for production of the culture product. Cells in the second continuous culture have a growth rate less than that of the first bioreactor. The growth rate of cells in the second bioreactor can be no more than any of 50%, 40%, 30%, 20%, 10%, 5%, 3%, 1%, 0.5%, or 0.1% that of the first bioreactor.

The culture conditions can be growth limiting. For example, the culture can comprise a growth limiting concentration of one or more nutrients. A growth limiting concentration is a concentration that prevents cells from growing at a maximal growth rate. The growth limiting doubling time of cells in the second bioreactor can be at least any of 12 hours, 24 hours, 48 hours, 72 hours, 96 hours, five days, six days, seven days, two weeks or one month. Alternatively, the growth limiting growth rate can be no more than any of 50%, 40%, 30%, 20%, 10%, 5%, 3%, 1%, 0.5%, or 0.1% of the maximum growth rate of the cells. Under limiting growth conditions, the opportunity for the development and growth of undesirable mutant microorganisms is decreased. This allows the second bioreactor to continue producing product for longer periods of time. Furthermore, by maintaining slow growth rates in the second continuous culture, the provided methods contribute to strain stability by decreasing the probability that mutant strains or contaminants will overtake the primary strains.

In the second bioreactor or bioreactors, cells typically are grown under constant culture conditions that produce, e.g., are optimized for, the production of a desired culture product. Optimal conditions for producing a product are specific to the product and the cells producing them. Methods for determining optimal culture conditions include, for example, varying culture parameters in identifying values or setpoints for the parameters that optimize product production. Culture parameters to be optimized include any of those described herein including, without limitation, temperature, pH, dissolved oxygen, and concentration of nutrients such as carbon sources, nitrogen sources, minerals, etc.

In certain embodiments, the methods provided herein comprise maintaining a constant growth rate in the second cell culture. Any suitable method for maintain a constant growth rate can be used, for example limiting one or more reagents/substrates/nutrients and/or adjusting the temperature.

In the second bioreactor, one or more nutrients are typically in growth-limiting concentrations, and also in relative concentration less than that of the first bioreactor. For example, the nutrient can be present in the first bioreactor at a concentration, e.g., molar concentration, that is at least any of 2 times, 4 times, ten times, 25 times, 50 times, 100 times or 500 times greater than the concentration of the nutrient in the second bioreactor.

In one embodiment the relative concentration of the rate limiting nutrient can be normalized against the amount of carbon in the first and second bioreactors, respectively. This can be useful when the concentration of carbon in the second bioreactor is not, itself, limiting. For example, the relative amount of carbon:rate limiting nutrient (e.g., phosphorous) can be 100:1 in the first bioreactor, but 1000:1 in the second bioreactor, a normalized ratio of 1:10. Accordingly, the normalized ratio of carbon:rate limiting nutrient in the first continuous culture to that in the second continuous culture can be at least any of 1:5, 1:10, 1:50, 1:100; 1:500; 1:1000; 1:5000 or 1:10,000.

In certain embodiments, the methods provided herein comprise maximizing bioproduction of the one or more culture products in the second bioreactor. In certain embodiments, bioproduction is performed in the one or more second bioreactors for at least about any of 10, 20, 30, 40, 80, 200, 250, 500, 1,000, 2,000, 3,000, 4,000, 5,000, 6,000, 7,000, 8,000, or 9,000 hours and/or not more than about any of 20, 30, 40, 80, 200, 250, 500, 1,000, 2,000, 3,000, 4,000, 5,000, 6,000, 7,000, 8,000, 9,000, or 10,000 hours, for example 1-10,000 hours, preferably 50-1,000 hours.

### 1. Cell Recycling

Cell cultures in the one or more second bioreactors typically are maintained at a constant volume. This necessitates removing culture fluid from the second bioreactor as culture fluid from the first bioreactor and other fluids, such as reagent fluids are added to the second bioreactor. Such removed fluids comprise cells being grown in the second bioreactor. In certain embodiments the cells can be returned to the second bioreactor. One method to do this is the use of a filter, such as a hollow fiber cartridge. Fluid entering one end of the hollow fiber cartridge is forced through the filter. However, cells are too large to pass through the filter. Accordingly, fluid-containing cells is returned to the second bioreactor. Fluid depleted from cells can be deposited in the container for product harvest.

### F. Product Harvest

Culture fluid in one or more of the second bioreactors (and optionally the first bioreactor depending on the metabolic pathways in the cell culture) contains a culture product for harvest. In certain embodiments, the method further comprises collecting/recovering the one or more culture products. In certain embodiments, the culture fluid from the one or more second bioreactors are removed from the chamber and the cells are separated from the culture fluid to produce a cell-free broth. In certain embodiments, the cell-free broth is continuously provided to a reservoir using a cell-recycling system.

Isolation of the product from the culture fluid depends on the nature of the product. For example, if the product is a protein, it can be isolated on an affinity column, for example comprising solid supports derivatized with an antibody that binds to the protein. Any suitable chromatography and/or separation method can be used, for example, size exclusion, ion exchange, or affinity chromatography. Small molecule products can be isolated from the culture fluid using chromatography, for example preparative HPLC, distillation, and/or crystallization. Any suitable method can be used.

### Exemplary Embodiments

1. A method comprising:
a) performing a first continuous culture of cells in a chamber of at least one first bioreactor under culture conditions that produce cell growth;
b) during the first continuous culture, moving culture fluid comprising cells from the chamber of the at least one first bioreactor into a chamber of at least one second bioreactor though one or more fluidic conduits that put the chamber of the at least one first bioreactor in fluidic communication with the chamber of the at least one second bioreactor; and
c) performing, in the chamber of the at least one second bioreactor, a second continuous culture of cells moved from the at least one first bioreactor, under culture conditions that produce at least one culture product;
wherein the rate of cell growth in the first continuous culture is greater than that of the second continuous culture, and the production of culture product in the first continuous culture is less than that of the second continuous culture.

2. The method of embodiment 1, further comprising:
d) collecting the at least one culture product from the chamber of the at least one second bioreactor.

3. The method of embodiment 1, wherein the rate of cell growth in the first continuous culture is at least twice that of the second continuous culture, and the production of culture product in the second continuous culture is at least twice that of first continuous culture.

4. The method of embodiment 1, wherein the rate of cell growth in the first continuous culture is at least 10 times that of the second continuous culture, and the production of culture product in the second continuous culture is at least 10 times that of first continuous culture.

5. The method of embodiment 1, wherein the rate of cell growth in the first continuous culture is at least 100 times that of the second continuous culture, and the production of culture product in the second continuous culture is at least 100 times that of first continuous culture.

6. The method of embodiment 1, wherein the rate of cell growth in the first continuous culture is at least 80%, e.g. at least 90%, of the maximum growth rate.

7. The method of embodiment 1, wherein the production of culture product in the second continuous culture is at least 80%, e.g. at least 90%, of the optimal culture product production rate.

8. The method of embodiment 1, comprising maintaining cell density in the at least one first bioreactor.

9. The method of embodiment 8, wherein maintaining cell density in the at least one first bioreactor comprises maintaining optical density of the first continuous cell culture.

10. The method of embodiment 8, wherein maintaining cell density comprises adding nutrient liquid to the first continuous culture.

11. The method of embodiment 8, wherein performing the first continuous culture further comprises maintaining constant volume of the culture of cells in the chamber of the at least one first bioreactor.

12. The method of embodiment 11, wherein maintaining volume comprises moving the culture fluid comprising media and cells from the chamber of the at least one first bioreactor to the chamber of the at least one second bioreactor when the volume of the first continuous culture exceed the target volume.

13. The method of embodiment 1, wherein moving the culture fluid from the chamber of the at least one first bioreactor to the chamber of the at least one second bioreactor comprises pumping the culture fluid with a pump.

14. The method of embodiment 1, comprising growing the cells in the chamber of the at least one first bioreactor at a constant cell growth rate.

15. The method of embodiment 1, wherein the growth rate of cells in the first continuous culture is at least any of 10%, 20%, 30%, 40%, 50%, 60%, 70%, 80%, 90% or 95% of a maximum cell growth rate.

16. The method of embodiment 1, wherein no nutrient in the first continuous culture is growth limiting.

17. The method of embodiment 1, wherein at least one nutrient, e.g., carbon or phosphorus, in the first continuous culture is at growth limiting concentration.

18. The method of embodiment 1, wherein the first continuous cell culture produces no culture product or minimal amounts of culture product.

19. The method of embodiment 1, wherein the rate of culture product production per unit volume in first continuous cell culture is less than any of 50%, 40%, 30%, 20%, 10%, 5%, 3%, or 1% that of the second continuous culture.

20. The method of embodiment 1, wherein the volume of the first continuous culture is less than that of the second continuous culture, e.g., wherein the relative volume proportion is less than any of 1:1, 1:10, 1:100, and 1:1000, e.g., between about 1:10 and 1:50.

21. The method of embodiment 1, wherein the volume of either of both of the first continuous culture and the second continuous culture is between about 50 ml and about 100,000 L, e.g., between about 500 mL and about 10 L.

22. The method of embodiment 1, wherein cells are moved from the chamber of the at least one first bioreactor during growth, e.g., log phase growth.

23. The method of embodiment 1, wherein culture fluid comprising cells is moved from the chamber of the at least one first bioreactor into a chamber of the at least one second bioreactor continuously over a period of at least any of one minute, five minutes, 10 minutes, 30 minutes, one hour, three hours, six hours, 12 hours, one day, two days, four days, one week, two weeks, one month, two months, three months, four months, five months, six months, seven months, eight months, nine months, ten months, eleven months or twelve months.

24. The method of embodiment 1, wherein culture fluid comprising cells is moved from the chamber of the at least one first bioreactor into a chamber of the at least one second bioreactor in a total volume of at least any of 0.01 liters, 0.1 liters, 1 L, 2 L, 5 L, 10 L, 50 L, 100 L, 500 L, 1,000 L, 2,000 L, 5,000 L, 10,000 L, 20,000 L, 50,000 L, 100,000 L, 500,000 L, 1,000,000 L, 5,000,000 L, 10,000,000 L, 50,000,000 L, 100,000.000 L and 1,000,000,000 L.

25. The method of embodiment 1, wherein culture fluid comprising cells is moved from the chamber of the at least one first bioreactor into a chamber of the at least one second bioreactor when the optical density (O.D.) of the first continuous culture reaches at least any of 1, 5, 10, 25, 50, 100, 200, and 400.

26. The method of embodiment 1, wherein the growth rate of cells in the second continuous culture is no more than any of 50%, 40%, 30%, 20%, 10%, 5%, 3%, or 1% that of the first continuous culture.

27. The method of embodiment 1, wherein the growth rate of cells in the second continuous culture is no more than any of 50%, 40%, 30%, 20%, 10%, 5%, 3%, or 1% of the maximum growth rate of the cells under non-limiting culture conditions.

28. The method of embodiment 1, wherein cells in the second continuous cell culture are not growing.

29. The method of embodiment 1, wherein at least one nutrient in the second continuous culture is present at a growth rate limiting concentration.

30. The method of embodiment 29, wherein the concentration of the nutrient in the first continuous culture is at least any of 2 times, 4 times, ten times, 25 times, 50 times, 100 times or 500 times greater than the concentration of the nutrient in the second continuous culture.

31. The method of embodiment 1, wherein the culture conditions of the second continuous culture comprise a growth limiting concentration of one or more nutrients (e.g., selected from metals (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), a salt, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol, amino acids (e.g., glutamate, leucine, and tryptophan), nucleic acid bases (e.g., adenine, cytosine, thymine, uracil, and guanine), or complex nutrients (e.g., yeast extract, peptone, tryptone, casamino acids and corn steep liquor)).

32. The method of embodiment 1, wherein the culture conditions in the second continuous culture are maintained constant for at least any of one hour, three hours, six hours, 12 hours, one day, two days, four days, one week, two weeks, one month, two months, three months, four months, five months, six months, seven months, eight months, nine months, ten months, eleven months or twelve months.

33. The method of embodiment 1, wherein the second continuous culture is performed for at least about any of 10, 20, 30, 40, 80, 200, 250, 500, 1,000, 2,000, 3,000, 4,000, 5,000, 6,000, 7,000, 8,000, or 9,000 hours and/or not more than about any of 20, 30, 40, 80, 200, 250, 500, 1,000, 2,000, 3,000, 4,000, 5,000, 6,000, 7,000, 8,000, 9,000, or 10,000 hours, for example 1-10,000 hours, preferably 50-1,000 hours.

34. The method of embodiment 1, comprising performing the second continuous culture in a plurality of different second bioreactors.

35. The method of embodiment 1, wherein the at least one first bioreactor is a plurality of first bioreactors.

36. The method of embodiment 1, wherein the culture conditions of the second continuous culture are optimized for production of at least one culture product.

37. The method of embodiment 1, wherein the culture conditions of the second continuous culture are different than culture conditions of the first continuous culture.

38. The method of embodiment 1, comprising, in the first and/or second continuous culture, maintaining one or more of a target pH, a target temperature, a target dissolved oxygen content, a target carbon concentration, and a target nitrogen concentration.

39. The method of embodiment 1, wherein culture conditions of the first and/or second continuous culture comprise maintaining constant concentration of one or more nutrients in the culture.

40. The method of embodiment 1, comprising inducing activity of a biochemical pathway that produces a culture output in the second continuous culture but not in the first continuous culture.

41. The method of embodiment 1, wherein the second continuous culture has an OD of at least any of 10, 50, 100 or 1000.

42. The method of embodiment 1, wherein cells in the second continuous culture have a doubling time of no more than any of 24 hours, 48 hours, 72 hours, 96 hours, 120 hours, five days, six days, seven days, two weeks, or one month.

43. The method of embodiment 1, wherein the cells in the second continuous culture are maintained at a constant density.

44. The method of embodiment 1, wherein cells removed from the chamber of the at least one second bioreactor are recycled into that chamber with a cell recycling device such as a hollow fiber filter.

45. The method of embodiment 1, wherein performing either first or second continuous culture comprises providing one or more of: a metal (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), a contamination control agent such as an antibiotic or biocide, an inducer, a salt, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol, antifoaming agents (e.g., antifoam 204, antifoam A, antifoam C), amino acids (e.g., glutamate, leucine, and tryptophan), nucleic acid bases (e.g., adenine, cytosine, thymine, uracil, and guanine), complex nutrients (e.g., yeast extract, peptone, tryptone, casamino acids and corn steep liquor), a macro-nutrient, a micro-nutrient, and a cell growth factor.

46. The method of embodiment 1, wherein performing either first or second continuous culture comprises providing a carbon source (e.g., a sugar (e.g., glucose, xylose, sucrose, glycerol, or acetate), molasses, malt extract, starch, dextrin, fruit pulp, CO or CO2).

47. The method of embodiment 1, wherein performing either first or second continuous culture comprises providing one or more of: a nitrogen source (e.g., amino acids or polypeptide, urea, ammonium salt (e.g., ammonium sulphate, ammonium phosphate or ammonia), corn steep liquor, yeast extract, peptone, and soybean meal).

48. The method of embodiment 1, wherein performing either first or second continuous culture comprises regulating one or more of: a nitrogen sparing rate, an aeration rate, an oxygen sparging rate, a carbon dioxide sparing rate, culture agitation speed, concentration of CO2, concentration of carbon source, concentration of nitrogen source, concentration of metals, vitamins, salts and concentration of an antibiotic.

49. The method of embodiment 1, wherein collecting the at least one culture product comprises removing cell culture fluid from the chamber of the at least one second bioreactor, and separating cells from the cell culture fluid to produce a cell free broth.

50. The method of embodiment 1, wherein a culture product is selected from a polypeptide (e.g., proteins, enzymes, antibodies), an organic molecule that is the product of a synthetic pathway in the cell (e.g., an industrial chemical such as a flavoring (e.g., vanillin); a flagrance (e.g., aldehydes, coumarins, indoles), an amino acid, an organic acid (e.g., citric, lactic and acetic acids); an alcohol (e.g., ethanol, isopropanol, ketones such as acetone); and a fatty acid (e.g., palmitic and oleic acid).

51. The method of embodiment 1, wherein the cells comprise archaea, prokaryotes and/or eukaryotes.

52. The method of embodiment 1, wherein the cells comprise fungal cells (e.g., yeast (e.g., Saccharomyces spp, Pichia spp, Komagataella spp, Kuyveromyces spp, Aspergillus spp, Rhodoporidium spp, Lipolytica spp, Aspergillus spp, Neurospora spp Trichoderma spp, Candida spp, or Penicillium).

53. The method of embodiment 1, wherein the cells comprise bacterial cells (e.g., Escherichia coli, Bacillus spp, Costridia spp, Streptomyces spp, Pseudomonas spp, Ralstonia spp, Shewanella spp).

54. The method of embodiment 1, wherein the cells comprise insect cells, animal cells or plant cells.

55. The method of embodiment 1, wherein the cells comprise animal cells (e.g., arthropods (e.g., insects, shrimp, lobster, crayfish and crabs); chordates (e.g., fish, amphibians, reptiles, birds (e.g., chickens or turkeys); mammals (e.g., human or non-human such as bovine, lamb, goat, pig, horse, dog, cat, primate)).

56. The method of embodiment 1, wherein the cells comprise a cell line (e.g., CHO (Chinese Hamster Ovary cells), BHK21 (Baby Hamster Kidney), NS0, Sp2/0 Murine Cell lines, insect cells (e.g., SP9, Sf9, sf21, S2) tobacco BY-2 cells, Oryza Sativa or algal cells).

57. The method of embodiment 1, wherein cells are not photosynthetic cells.

58. A system comprising:
(a) at least one first bioreactor comprising:
   (i) a first chamber; and
   (ii) one or more first sensors providing at least a measure of cell density and a measure of volume of a cell culture in the first chamber;
(b) at least one second bioreactor comprising:
   (i) a second chamber; and
   (ii) one or more second sensors providing at least a measure of culture conditions and a measure of volume of a cell culture in the one or more second chambers;
wherein the first chamber is in fluidic communication with the one or more second chambers;
(c) one or more reagent reservoirs comprising liquid reagents, wherein the reagent reservoirs are in fluidic communication with the first chamber and, optionally, with the one or more second chambers;
(d) one or more pumps configured to:
   (i) move liquid reagents from at least one reagent reservoir to the first chamber;
   (ii) move cell culture fluid from the first chamber to the one or more second chambers;
   (iii) move cell culture fluid out of the one or more second chambers;
   (iv) optionally move liquid reagents from at least one reagent reservoir to the one or more second chambers; and
(e) a control system configured to:
   (i) use measures from the one or more sensors to control cell growth, e.g., cell density, of a culture of cells in the first chamber at a rate greater than that of the second chamber;
   (ii) move cell culture fluid from the first chamber to the one or more second chambers; and
   (iii) use measures from the one or more sensors to control culture conditions and, e.g., volume and/or cell growth, of a culture of cells in the one or more second chambers to produce a culture product at a rate greater than that of the first chamber.

59. The method of embodiment 58, wherein the control system uses measures from the one or more sensors to establish a rate of cell growth in the first continuous culture is at least twice, at least 10 times or at least 100 times that of the second continuous culture, and a rate of production of culture product in the second continuous culture is at least twice, at least 10 times or at least 100 times that of first continuous culture.

60. The system of embodiment 58, further comprising one or more collection vessels in fluid communication with the one or more second chambers.

61. The system of embodiment 58, wherein the first chamber and the one or more second chambers have volumes between 250 ml and 1,000,000 liters.

62. The system of embodiment 58, wherein the control system sets a dilution rate of liquid reagent being moved into the first and/or one or more second chambers to maintain concentration of one or more nutrients.

63. The system of embodiment 58, wherein the first chamber and the one or more second chambers comprise ports that communicate with reagent reservoirs and with each other through fluidic conduits.

64. The system of embodiment 58, wherein the one or more second chambers is a plurality of second chambers.

65. The system of embodiment 58, wherein one or more first or second sensors providing a measure of volume comprise a scale measuring mass of a bioreactor.

66. The system of embodiment 58, wherein a sensor providing a measure of cell density of a cell culture in the first chamber comprises an optical density or capacitance sensor.

67. The system of embodiment 65, wherein controlling cell density comprises controlling optical density of the cell culture.

68. The system of embodiment 66, comprising a user-programmable module that computes a dilution rate as a function of error between target optical density and measured optical density.

69. The system of embodiment 58, wherein the one or more first sensors provide measures of temperature, pH and dissolved oxygen of a cell culture in the first chamber.

70. The system of embodiment 58, wherein the one or more second sensors provide measures of temperature, pH and dissolved oxygen of a cell culture in the second chamber.

71. The system of embodiment 58, wherein at least one pump is a peristaltic pump or a gravity pump.

72. The system of embodiment 70, wherein the peristaltic pump comprises a fitting for a tube that fluidically communicates between two chambers, a chamber and a reagent reservoir, or the second chamber and an effluent conduit.

73. The system of embodiment 58, wherein the control system that controls volume of a cell culture in the first chamber or the one or more second chambers comprises a level sensor (e.g., an overflow tube) that moves cell culture fluid from a chamber when the height of the cell culture exceeds the top of the level sensor.

74. The system of embodiment 58, wherein the control system comprises a computer comprising a processor and memory comprising executable code which, when executed by the processor, performs one or more feedback routines to control the cell growth, e.g., cell density, in the first chamber, control volume of a cell culture in the first chamber and/or one or more second chambers, and control the culture conditions in the one or more second chambers control.

75. The system of embodiment 73, wherein one of the feedback routines calculates the cell density of a cell culture in the first chamber based on a measure received from the one or more sensors, and actuates a pump to move liquid reagent from a reagent reservoir to the first chamber to adjust the cell density to a set point.

76. The system of embodiment 73, wherein one of the feedback routines calculates the volume of a cell culture in the first chamber based on a measure received from the one or more sensors, and, if the volume is above a set point, actuates a pump to move liquid cell culture from the first chamber to the one or more second chambers.

77. The system of embodiment 73, wherein:
(I) the one or more sensors provide measures of one or more culture parameters in the first and/or one or more second chambers;
(II) the system further comprises one or more effectors to affect changes in the one or more culture parameters;
(III) one or more one of the feedback routines calculate one or more culture parameters based on the measures, and actuate the one or more effectors to adjust the culture parameters toward target levels.

78. The system of embodiment 73, wherein:
(I) the one or more sensors provide measures of one or more of pH, temperature and dissolved O2 of a cell culture in the first and/or second one or more chambers;
(II) the system further comprises one or more of:
   one or more reagent reservoirs comprising an acid and a base in fluid communication with the first and/or one or more second reservoirs;
   one or more temperature controllers to control temperature of a cell culture in the first and/or one or more second chambers;
   one or more aerators for aerating a cell culture in the first chamber and/or one or more second chambers;
(III) one or more one of the feedback routines calculate a culture condition comprising one or more of pH, temperature, dissolved 02, and nutrient concentration based on a measure received from the one or more sensors, and actuates:
   one or more pumps to move acid or base from the reagent reservoir into the second chamber, to adjust pH to a target pH;
   one or more temperature controllers to adjust temperature to a target temperature; and/or
   one or more aerators to aerate the cell culture to a target dissolved oxygen level.

79. The system of embodiment 73, wherein one feedback routine calculates the volume of a cell culture in the one or more second chambers based on a measure received from the one or more sensors, and, if the volume is above a set point, actuates a pump to move liquid cell culture from the one or more second chambers.

80. The system of embodiment 58, further comprising a filter in fluid communication with an exit of the second chamber, configured to recycle cells that cannot pass through a membrane back into the second chamber, and passes cell-depleted liquid culture medium out of the cartridge to a collection vessel.

81. The system of embodiment 79, wherein the filter comprises a hollow fiber cartridge.

82. The system of embodiment 58, wherein the first chamber comprises a cell culture, and wherein the one or more reagent reservoirs in fluidic communication with the first chamber comprise growth medium in which one or more nutrients are present at a concentration that limits growth rate of the cells in the cell culture.

83. The system of embodiment 58, wherein the culture media is formulated such that nutrient is not limited in the at least one first bioreactor but one or more nutrients is present at a concentration that limits growth in the one or more second bioreactors.

84. The system of embodiment 58, wherein the one or more control subsystems comprise a computer comprising:
(A) a processor;
(B) a memory coupled to the processor, and
(C) computer executable instructions that use measures from the one or sensors to calculate culture conditions in the first chamber and/or one or more second chambers, based on the calculations, control the pumps to move liquids to and from the chambers.

85. The system of embodiment 58, comprising one or more of:
a temperature sensor and a temperature regulator;
a dissolved oxygen meter and an aeration system communicating with an interior of the first and/or one or more second chambers;
an analyzer for measuring concentration of a nutrient in a cell culture in a chamber;
an impeller or a pneumatic agitator to mix liquid in a chamber, and a motor configured to actuate the impeller or pneumatic agitator;
an effluent communicating with the vessel interior and a regulatable valve or pump for regulating fluid flow from the chamber;
baffles in the chamber;
a sparger and mass flow controller communicating with the chamber interior for input of one or more gases and mixtures thereof;
a user interface for communicating instructions with the computer;
a foam control;
a cell recycling hollow fiber membrane; and
a reverse or forward osmosis membrane.

86. The system of embodiment 58, wherein the control system is configured to carry out any of the methods of embodiments 1-35.

87. A method comprising:
a) performing a first continuous culture of cells in a chamber of a first bioreactor under culture conditions that maintain a constant cell density;
b) during the first continuous culture, moving culture fluid comprising cells from the chamber of the first bioreactor into a chamber of at least one second bioreactor though one or more fluidic conduits that put the chamber of the first bioreactor in fluidic communication with the chamber of the at least one second bioreactor;
c) performing a second continuous culture of cells moved from the first bioreactor in the chamber of each of the one or more second bioreactors under constant culture conditions, to produce at least one culture product; and
d) collecting the at least one culture product from the chamber of each of the one or more second bioreactors.

88. A system comprising:
(a) a first bioreactor comprising:
   (i) a first chamber; and
   (ii) one or more first sensors providing at least a measure of cell density and a measure of volume of a cell culture in the first chamber;
(b) one or more second bioreactors comprising:
   (i) a second chamber; and
   (ii) one or more second sensors providing at least a measure of culture conditions and a measure of volume of a cell culture in the one or more second chambers;
wherein the first chamber is in fluidic communication with the one or more second chambers;
(c) one or more reagent reservoirs comprising liquid reagents, wherein the reagent reservoirs are in fluidic communication with the first chamber and, optionally, with the one or more second chambers;
(d) one or more pumps configured to:
   (i) move liquid reagents from at least one reagent reservoir to the first chamber;
   (ii) move cell culture fluid from the first chamber to the one or more second chambers;
   (iii) move cell culture fluid out of the one or more second chambers;
   (iv) optionally move liquid reagents from at least one reagent reservoir to the one or more second chambers; and
(e) a control system configured to:
   (i) use measures from the one or more sensors to control cell growth, e.g., cell density, of a culture of cells in the first chamber;
   (ii) move cell culture fluid from the first chamber to the one or more second chambers; and
   (iii) use measures from the one or more sensors to control culture conditions and, e.g., volume and/or cell growth, of a culture of cells in the one or more second chambers.

### EXAMPLES

### Example 1: Production of a Culture Product Using a Multi-Chamber Cell Culture System

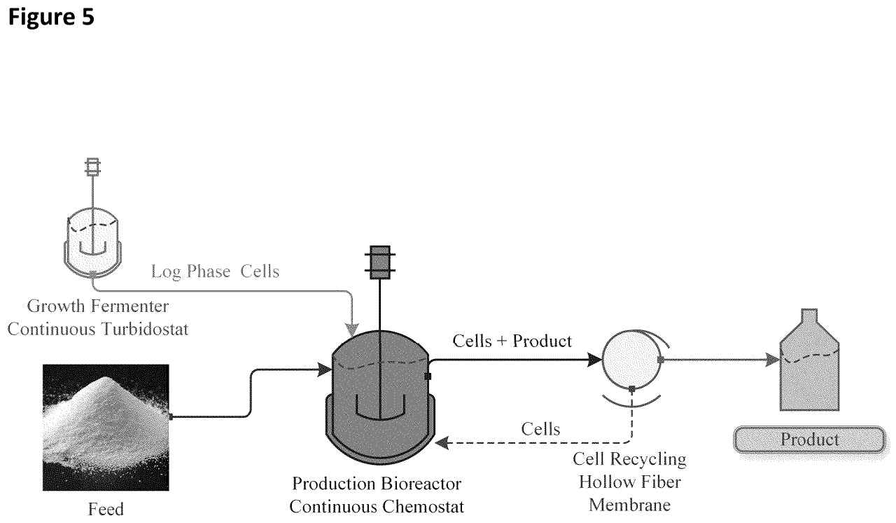

This example demonstrates the ability to produce a culture product using a multi-chamber cell culture system (FIG. 5).

Inoculum Preparation: Bacterial colonies were streaked onto LB agar plates with appropriate antibiotics from a glycerol stock stored at −80° C. and grown under suitable conditions until visible colonies formed. 5 mL of growth medium with appropriate antibiotics was inoculated with a single colony from the LB agar plate and grown overnight at 37° C. with shaking at 250 rpm. The following day, 300 mL of growth medium was inoculated with 3 mL of the turbid overnight cultures and incubated at 37° C. with shaking at 250 rpm. When the optical density of the culture at 600 nm reached approximately 1, the cells were ready for inoculation into the first bioreactor. 10 mL aliquots of cells from the 300 mL culture were stored in 15% glycerol in a −80° C. freezer for future bioreactor runs.

The following bioreactor runs were performed using Sartorius Biostat B 1.8 L bioreactors.

Bioreactor Assembly and Sterilization: Two bioreactors were prepared. The reactor vessel head plate was secured to the glass vessel and the following components were inserted into the vessel head ports: (1) a condenser, (2) a sparger, (3) a 4 way port assembly (1 port for feed, 2 ports for acid/base, 1 port for additions), (4) a sampling port, (5) a effluent port, (6) a dissolved oxygen (DO) probe, and (7) a pH probe. The assembly was autoclaved for 30 minutes at 121° C. along with feed reservoir bottles, growth medium, and feed tubing. After autoclaving and the vessel was cooled to room temperature, an OD probe was mounted to the appropriate vessel head port to measure optical density. The bioreactor was set on a scale and the scale zeroed after putting in the batch media (500-1000 L). The base bottle was connected to both tanks. The growth medium was set on a scale attached to the first bioreactor. The effluent port of the first bioreactor was connected to the second bioreactor. A glucose reservoir was connected to the second bioreactor. The effluent from the second bioreactor was connected to a 0.2 um hollow fiber cell recycling system, and the maximum back and forward pressure was set to <30 psi. The filtrate end of the 0.2 um hollow fiber cell recycling system was connected to a collection reservoir on a scale, and the retentate end of the 0.2 um hollow fiber cell recycling system was connected to an inlet of the second bioreactor.

Bioreactor Operation: After assembly, any post sterilization addition reagents were added to the growth medium. All the components of the bioreactor were connected to the Biostat-B control system and the temperature probe was inserted. This allowed the Fermwork software to receive inputs from the temperature, DO, and pH probes and to control agitation, airflow, temperature, and feed & base pumps. The feed and base lines were connected from their respective reservoirs and into the 3-way port assembly. Pump tubing section of lines were inserted into the appropriate pump heads, and the lines were primed by manually running pumps until liquid was seen entering the bioreactor, and then switch pumps back to automated control. The base reservoir contained 14-28% ammonium hydroxide.

The system was then allowed to stabilize with the following parameters: 37° C., pH: 6.8; DO: 30% of saturation. Once stabilized, the first bioreactor was inoculated to a starting OD₆₀₀ of 0.05-0.3 by injecting ˜10 mL seed culture into the addition port using a sterile syringe. The turbidostat program was initiated in first bioreactor after the cells reaches mid-log phase. This allows a constant biomass concentration in first bioreactor and pumps the extra cells to the second bioreactor. The mass_stat program on first bioreactor was initiated to maintain constant volume with the effluent pump speed controlled by weight of the tank mass. The second bioreactor was initiated as a standard fed-batch reactor. The feed is triggered by a hunger spike, a sharp rising in dissolved 02 indicating carbon source limitation. After the first bioreactor and the second bioreactor stabilized, the pumps that move effluent from the second bioreactor to the hollow fiber cell recycling system were then turned on, this allows cells to be enriched and recycled in the second bioreactor. The mass_stat program was then initiated on the second bioreactor where the effluent pump to the hollow fibers is controlled by mass of the second bioreactor tank. Optionally, a purge tank is connected to the second bioreactor via a dip tube to collect fluid and cells from second bioreactor above a set volume in the second bioreactor. The purge tank is placed on a balance to measure mass of purge fluid removed.

Samples were periodically taken for analysis from the first bioreactor, the second bioreactor, and the second bioreactor effluent tank, the second bioreactor purge tank. The OD of the samples was measured spectroscopically, and the supernatant was analyzed by HPLC for the culture product and mass of the tanks recorded to calculate culture product production and carbon source consumption.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “about” refers to a range that is 5% plus or minus from a stated numerical value within the context of the particular usage. So, for example, “about 100” means between 95 and 105. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
a) performing a first continuous culture of cells in a chamber of at least one first bioreactor under culture conditions that produce cell growth;
b) during the first continuous culture, moving culture fluid comprising cells from the chamber of the at least one first bioreactor into a chamber of at least one second bioreactor though one or more fluidic conduits that put the chamber of the at least one first bioreactor in fluidic communication with the chamber of the at least one second bioreactor; and
c) performing, in the chamber of the at least one second bioreactor, a second continuous culture of cells moved from the at least one first bioreactor, under culture conditions that produce at least one culture product;
wherein the rate of cell growth in the first continuous culture is greater than that of the second continuous culture, and the production of culture product in the first continuous culture is less than that of the second continuous culture.

2. The method of claim 1, further comprising:
d) collecting the at least one culture product from the chamber of the at least one second bioreactor.

3. The method of claim 1, wherein the rate of cell growth in the first continuous culture is at least twice that of the second continuous culture, and the production of culture product in the second continuous culture is at least twice that of first continuous culture.

4. The method of claim 1, wherein the rate of cell growth in the first continuous culture is at least 10 times that of the second continuous culture, and the production of culture product in the second continuous culture is at least 10 times that of first continuous culture.

5. The method of claim 1, wherein the rate of cell growth in the first continuous culture is at least 100 times that of the second continuous culture, and the production of culture product in the second continuous culture is at least 100 times that of first continuous culture.

6. The method of claim 1, wherein the rate of cell growth in the first continuous culture is at least 80%, e.g. at least 90%, of the maximum growth rate.

7. The method of claim 1, wherein the production of culture product in the second continuous culture is at least 80%, e.g. at least 90%, of the optimal culture product production rate.

8. The method of claim 1, comprising maintaining cell density in the at least one first bioreactor.

9. The method of claim 8, wherein maintaining cell density in the at least one first bioreactor comprises maintaining optical density of the first continuous cell culture.

10. The method of claim 8, wherein maintaining cell density comprises adding nutrient liquid to the first continuous culture.

11. The method of claim 8, wherein performing the first continuous culture further comprises maintaining constant volume of the culture of cells in the chamber of the at least one first bioreactor.

12. The method of claim 11, wherein maintaining volume comprises moving the culture fluid comprising media and cells from the chamber of the at least one first bioreactor to the chamber of the at least one second bioreactor when the volume of the first continuous culture exceed the target volume.

13. The method of claim 1, wherein moving the culture fluid from the chamber of the at least one first bioreactor to the chamber of the at least one second bioreactor comprises pumping the culture fluid with a pump.

14. The method of claim 1, comprising growing the cells in the chamber of the at least one first bioreactor at a constant cell growth rate.

15. The method of claim 1, wherein the growth rate of cells in the first continuous culture is at least any of 10%, 20%, 30%, 40%, 50%, 60%, 70%, 80%, 90% or 95% of a maximum cell growth rate.

16. The method of claim 1, wherein no nutrient in the first continuous culture is growth limiting.

17. The method of claim 1, wherein at least one nutrient, e.g., carbon or phosphorus, in the first continuous culture is at growth limiting concentration.

18. The method of claim 1, wherein the first continuous cell culture produces no culture product or minimal amounts of culture product.

19. The method of claim 1, wherein the rate of culture product production per unit volume in first continuous cell culture is less than any of 50%, 40%, 30%, 20%, 10%, 5%, 3%, or 1% that of the second continuous culture.

20. The method of claim 1, wherein the volume of the first continuous culture is less than that of the second continuous culture, e.g., wherein the relative volume proportion is less than any of 1:1, 1:10, 1:100, and 1:1000, e.g., between about 1:10 and 1:50.

21. The method of claim 1, wherein the volume of either of both of the first continuous culture and the second continuous culture is between about 50 ml and about 100,000 L, e.g., between about 500 mL and about 10 L.

22. The method of claim 1, wherein cells are moved from the chamber of the at least one first bioreactor during growth, e.g., log phase growth.

23. The method of claim 1, wherein culture fluid comprising cells is moved from the chamber of the at least one first bioreactor into a chamber of the at least one second bioreactor continuously over a period of at least any of one minute, five minutes, 10 minutes, 30 minutes, one hour, three hours, six hours, 12 hours, one day, two days, four days, one week, two weeks, one month, two months, three months, four months, five months, six months, seven months, eight months, nine months, ten months, eleven months or twelve months.

24. The method of claim 1, wherein culture fluid comprising cells is moved from the chamber of the at least one first bioreactor into a chamber of the at least one second bioreactor in a total volume of at least any of 0.01 liters, 0.1 liters, 1 L, 2 L, 5 L, 10 L, 50 L, 100 L, 500 L, 1,000 L, 2,000 L, 5,000 L, 10,000 L, 20,000 L, 50,000 L, 100,000 L, 500,000 L, 1,000,000 L, 5,000,000 L, 10,000,000 L, 50,000,000 L, 100,000.000 L and 1,000,000,000 L.

25. The method of claim 1, wherein culture fluid comprising cells is moved from the chamber of the at least one first bioreactor into a chamber of the at least one second bioreactor when the optical density (O.D.) of the first continuous culture reaches at least any of 1, 5, 10, 25, 50, 100, 200, and 400.

26. The method of claim 1, wherein the growth rate of cells in the second continuous culture is no more than any of 50%, 40%, 30%, 20%, 10%, 5%, 3%, or 1% that of the first continuous culture.

27. The method of claim 1, wherein the growth rate of cells in the second continuous culture is no more than any of 50%, 40%, 30%, 20%, 10%, 5%, 3%, or 1% of the maximum growth rate of the cells under non-limiting culture conditions.

28. The method of claim 1, wherein cells in the second continuous cell culture are not growing.

29. The method of claim 1, wherein at least one nutrient in the second continuous culture is present at a growth rate limiting concentration.

30. The method of claim 29, wherein the concentration of the nutrient in the first continuous culture is at least any of 2 times, 4 times, ten times, 25 times, 50 times, 100 times or 500 times greater than the concentration of the nutrient in the second continuous culture.

31. The method of claim 1, wherein the culture conditions of the second continuous culture comprise a growth limiting concentration of one or more nutrients (e.g., selected from metals (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), a salt, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol, amino acids (e.g., glutamate, leucine, and tryptophan), nucleic acid bases (e.g., adenine, cytosine, thymine, uracil, and guanine), or complex nutrients (e.g., yeast extract, peptone, tryptone, casamino acids and corn steep liquor)).

32. The method of claim 1, wherein the culture conditions in the second continuous culture are maintained constant for at least any of one hour, three hours, six hours, 12 hours, one day, two days, four days, one week, two weeks, one month, two months, three months, four months, five months, six months, seven months, eight months, nine months, ten months, eleven months or twelve months.

33. The method of claim 1, wherein the second continuous culture is performed for at least about any of 10, 20, 30, 40, 80, 200, 250, 500, 1,000, 2,000, 3,000, 4,000, 5,000, 6,000, 7,000, 8,000, or 9,000 hours and/or not more than about any of 20, 30, 40, 80, 200, 250, 500, 1,000, 2,000, 3,000, 4,000, 5,000, 6,000, 7,000, 8,000, 9,000, or 10,000 hours, for example 1-10,000 hours, preferably 50-1,000 hours.

34. The method of claim 1, comprising performing the second continuous culture in a plurality of different second bioreactors.

35. The method of claim 1, wherein the at least one first bioreactor is a plurality of first bioreactors.

36. The method of claim 1, wherein the culture conditions of the second continuous culture are optimized for production of at least one culture product.

37. The method of claim 1, wherein the culture conditions of the second continuous culture are different than culture conditions of the first continuous culture.

38. The method of claim 1, comprising, in the first and/or second continuous culture, maintaining one or more of a target pH, a target temperature, a target dissolved oxygen content, a target carbon concentration, and a target nitrogen concentration.

39. The method of claim 1, wherein culture conditions of the first and/or second continuous culture comprise maintaining constant concentration of one or more nutrients in the culture.

40. The method of claim 1, comprising inducing activity of a biochemical pathway that produces a culture output in the second continuous culture but not in the first continuous culture.

41. The method of claim 1, wherein the second continuous culture has an OD of at least any of 10, 50, 100 or 1000.

42. The method of claim 1, wherein cells in the second continuous culture have a doubling time of no more than any of 24 hours, 48 hours, 72 hours, 96 hours, 120 hours, five days, six days, seven days, two weeks, or one month.

43. The method of claim 1, wherein the cells in the second continuous culture are maintained at a constant density.

44. The method of claim 1, wherein cells removed from the chamber of the at least one second bioreactor are recycled into that chamber with a cell recycling device such as a hollow fiber filter.

45. The method of claim 1, wherein performing either first or second continuous culture comprises providing one or more of: a metal (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), a contamination control agent such as an antibiotic or biocide, an inducer, a salt, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol, antifoaming agents (e.g., antifoam 204, antifoam A, antifoam C), amino acids (e.g., glutamate, leucine, and tryptophan), nucleic acid bases (e.g., adenine, cytosine, thymine, uracil, and guanine), complex nutrients (e.g., yeast extract, peptone, tryptone, casamino acids and corn steep liquor), a macro-nutrient, a micro-nutrient, and a cell growth factor.

46. The method of claim 1, wherein performing either first or second continuous culture comprises providing a carbon source (e.g., a sugar (e.g., glucose, xylose, sucrose, glycerol, or acetate), molasses, malt extract, starch, dextrin, fruit pulp, CO or CO₂).

47. The method of claim 1, wherein performing either first or second continuous culture comprises providing one or more of: a nitrogen source (e.g., amino acids or polypeptide, urea, ammonium salt (e.g., ammonium sulphate, ammonium phosphate or ammonia), corn steep liquor, yeast extract, peptone, and soybean meal).

48. The method of claim 1, wherein performing either first or second continuous culture comprises regulating one or more of: a nitrogen sparing rate, an aeration rate, an oxygen sparging rate, a carbon dioxide sparing rate, culture agitation speed, concentration of CO₂, concentration of carbon source, concentration of nitrogen source, concentration of metals, vitamins, salts and concentration of an antibiotic.

49. The method of claim 1, wherein collecting the at least one culture product comprises removing cell culture fluid from the chamber of the at least one second bioreactor, and separating cells from the cell culture fluid to produce a cell free broth.

50. The method of claim 1, wherein a culture product is selected from a polypeptide (e.g., proteins, enzymes, antibodies), an organic molecule that is the product of a synthetic pathway in the cell (e.g., an industrial chemical such as a flavoring (e.g., vanillin); a flagrance (e.g., aldehydes, coumarins, indoles), an amino acid, an organic acid (e.g., citric, lactic and acetic acids); an alcohol (e.g., ethanol, isopropanol, ketones such as acetone); and a fatty acid (e.g., palmitic and oleic acid).

51. The method of claim 1, wherein the cells comprise archaea, prokaryotes and/or eukaryotes.

52. The method of claim 1, wherein the cells comprise fungal cells (e.g., yeast (e.g., Saccharomyces spp, Pichia spp, Komagataella spp, Kuyveromyces spp, Aspergillus spp, Rhodoporidium spp, Lipolytica spp, Aspergillus spp, Neurospora spp Trichoderma spp, Candida spp, or Penicillium).

53. The method of claim 1, wherein the cells comprise bacterial cells (e.g., Escherichia coli, Bacillus spp, Costridia spp, Streptomyces spp, Pseudomonas spp, Ralstonia spp, Shewanella spp).

54. The method of claim 1, wherein the cells comprise insect cells, animal cells or plant cells.

55. The method of claim 1, wherein the cells comprise animal cells (e.g., arthropods (e.g., insects, shrimp, lobster, crayfish and crabs); chordates (e.g., fish, amphibians, reptiles, birds (e.g., chickens or turkeys); mammals (e.g., human or non-human such as bovine, lamb, goat, pig, horse, dog, cat, primate)).

56. The method of claim 1, wherein the cells comprise a cell line (e.g., CHO (Chinese Hamster Ovary cells), BHK21 (Baby Hamster Kidney), NS0, Sp2/0 Murine Cell lines, insect cells (e.g., SP9, Sf9, sf21, S2) tobacco BY-2 cells, Oryza Sativa or algal cells).

57. The method of claim 1, wherein cells are not photosynthetic cells.

58. A system comprising:
(a) at least one first bioreactor comprising:
(i) a first chamber; and
(ii) one or more first sensors providing at least a measure of cell density and a measure of volume of a cell culture in the first chamber;
(b) at least one second bioreactor comprising:
(i) a second chamber; and
(ii) one or more second sensors providing at least a measure of culture conditions and a measure of volume of a cell culture in the one or more second chambers;
wherein the first chamber is in fluidic communication with the one or more second chambers;
(c) one or more reagent reservoirs comprising liquid reagents, wherein the reagent reservoirs are in fluidic communication with the first chamber and, optionally, with the one or more second chambers;
(d) one or more pumps configured to:
(i) move liquid reagents from at least one reagent reservoir to the first chamber;
(ii) move cell culture fluid from the first chamber to the one or more second chambers;
(iii) move cell culture fluid out of the one or more second chambers;
(iv) optionally move liquid reagents from at least one reagent reservoir to the one or more second chambers; and
(e) a control system configured to:
(i) use measures from the one or more sensors to control cell growth, e.g., cell density, of a culture of cells in the first chamber at a rate greater than that of the second chamber;
(ii) move cell culture fluid from the first chamber to the one or more second chambers; and
(iii) use measures from the one or more sensors to control culture conditions and, e.g., volume and/or cell growth, of a culture of cells in the one or more second chambers to produce a culture product at a rate greater than that of the first chamber.

59. The method of claim 58, wherein the control system uses measures from the one or more sensors to establish a rate of cell growth in the first continuous culture is at least twice, at least 10 times or at least 100 times that of the second continuous culture, and a rate of production of culture product in the second continuous culture is at least twice, at least 10 times or at least 100 times that of first continuous culture.

60. The system of claim 58, further comprising one or more collection vessels in fluid communication with the one or more second chambers.

61. The system of claim 58, wherein the first chamber and the one or more second chambers have volumes between 250 ml and 1,000,000 liters.

62. The system of claim 58, wherein the control system sets a dilution rate of liquid reagent being moved into the first and/or one or more second chambers to maintain concentration of one or more nutrients.

63. The system of claim 58, wherein the first chamber and the one or more second chambers comprise ports that communicate with reagent reservoirs and with each other through fluidic conduits.

64. The system of claim 58, wherein the one or more second chambers is a plurality of second chambers.

65. The system of claim 58, wherein one or more first or second sensors providing a measure of volume comprise a scale measuring mass of a bioreactor.

66. The system of claim 58, wherein a sensor providing a measure of cell density of a cell culture in the first chamber comprises an optical density or capacitance sensor.

67. The system of claim 65, wherein controlling cell density comprises controlling optical density of the cell culture.

68. The system of claim 66, comprising a user-programmable module that computes a dilution rate as a function of error between target optical density and measured optical density.

69. The system of claim 58, wherein the one or more first sensors provide measures of temperature, pH and dissolved oxygen of a cell culture in the first chamber.

70. The system of claim 58, wherein the one or more second sensors provide measures of temperature, pH and dissolved oxygen of a cell culture in the second chamber.

71. The system of claim 58, wherein at least one pump is a peristaltic pump or a gravity pump.

72. The system of claim 70, wherein the peristaltic pump comprises a fitting for a tube that fluidically communicates between two chambers, a chamber and a reagent reservoir, or the second chamber and an effluent conduit.

73. The system of claim 58, wherein the control system that controls volume of a cell culture in the first chamber or the one or more second chambers comprises a level sensor (e.g., an overflow tube) that moves cell culture fluid from a chamber when the height of the cell culture exceeds the top of the level sensor.

74. The system of claim 58, wherein the control system comprises a computer comprising a processor and memory comprising executable code which, when executed by the processor, performs one or more feedback routines to control the cell growth, e.g., cell density, in the first chamber, control volume of a cell culture in the first chamber and/or one or more second chambers, and control the culture conditions in the one or more second chambers control.

75. The system of claim 73, wherein one of the feedback routines calculates the cell density of a cell culture in the first chamber based on a measure received from the one or more sensors, and actuates a pump to move liquid reagent from a reagent reservoir to the first chamber to adjust the cell density to a set point.

76. The system of claim 73, wherein one of the feedback routines calculates the volume of a cell culture in the first chamber based on a measure received from the one or more sensors, and, if the volume is above a set point, actuates a pump to move liquid cell culture from the first chamber to the one or more second chambers.

77. The system of claim 73, wherein:
(I) the one or more sensors provide measures of one or more culture parameters in the first and/or one or more second chambers;
(II) the system further comprises one or more effectors to affect changes in the one or more culture parameters;
(III) one or more one of the feedback routines calculate one or more culture parameters based on the measures, and actuate the one or more effectors to adjust the culture parameters toward target levels.

78. The system of claim 73, wherein:
(I) the one or more sensors provide measures of one or more of pH, temperature and dissolved 02 of a cell culture in the first and/or second one or more chambers;
(II) the system further comprises one or more of:
one or more reagent reservoirs comprising an acid and a base in fluid communication with the first and/or one or more second reservoirs;
one or more temperature controllers to control temperature of a cell culture in the first and/or one or more second chambers;
one or more aerators for aerating a cell culture in the first chamber and/or one or more second chambers;
(III) one or more one of the feedback routines calculate a culture condition comprising one or more of pH, temperature, dissolved 02, and nutrient concentration based on a measure received from the one or more sensors, and actuates:
one or more pumps to move acid or base from the reagent reservoir into the second chamber, to adjust pH to a target pH;
one or more temperature controllers to adjust temperature to a target temperature; and/or
one or more aerators to aerate the cell culture to a target dissolved oxygen level.

79. The system of claim 73, wherein one feedback routine calculates the volume of a cell culture in the one or more second chambers based on a measure received from the one or more sensors, and, if the volume is above a set point, actuates a pump to move liquid cell culture from the one or more second chambers.

80. The system of claim 58, further comprising a filter in fluid communication with an exit of the second chamber, configured to recycle cells that cannot pass through a membrane back into the second chamber, and passes cell-depleted liquid culture medium out of the cartridge to a collection vessel.

81. The system of claim 79, wherein the filter comprises a hollow fiber cartridge.

82. The system of claim 58, wherein the first chamber comprises a cell culture, and wherein the one or more reagent reservoirs in fluidic communication with the first chamber comprise growth medium in which one or more nutrients are present at a concentration that limits growth rate of the cells in the cell culture.

83. The system of claim 58, wherein the culture media is formulated such that nutrient is not limited in the at least one first bioreactor but one or more nutrients is present at a concentration that limits growth in the one or more second bioreactors.

84. The system of claim 58, wherein the one or more control subsystems comprise a computer comprising:
(A) a processor;
(B) a memory coupled to the processor, and
(C) computer executable instructions that use measures from the one or sensors to calculate culture conditions in the first chamber and/or one or more second chambers, based on the calculations, control the pumps to move liquids to and from the chambers.

85. The system of claim 58, comprising one or more of:
a temperature sensor and a temperature regulator;
a dissolved oxygen meter and an aeration system communicating with an interior of the first and/or one or more second chambers;
an analyzer for measuring concentration of a nutrient in a cell culture in a chamber;
an impeller or a pneumatic agitator to mix liquid in a chamber, and a motor configured to actuate the impeller or pneumatic agitator;
an effluent communicating with the vessel interior and a regulatable valve or pump for regulating fluid flow from the chamber;
baffles in the chamber;
a sparger and mass flow controller communicating with the chamber interior for input of one or more gases and mixtures thereof;
a user interface for communicating instructions with the computer;
a foam control;
a cell recycling hollow fiber membrane; and
a reverse or forward osmosis membrane.

86. The system of claim 58, wherein the control system is configured to carry out any of the methods of claims 1-35.

87. A method comprising:
a) performing a first continuous culture of cells in a chamber of a first bioreactor under culture conditions that maintain a constant cell density;
b) during the first continuous culture, moving culture fluid comprising cells from the chamber of the first bioreactor into a chamber of at least one second bioreactor though one or more fluidic conduits that put the chamber of the first bioreactor in fluidic communication with the chamber of the at least one second bioreactor;
c) performing a second continuous culture of cells moved from the first bioreactor in the chamber of each of the one or more second bioreactors under constant culture conditions, to produce at least one culture product; and
d) collecting the at least one culture product from the chamber of each of the one or more second bioreactors.

88. A system comprising:
(a) a first bioreactor comprising:
(i) a first chamber; and
(ii) one or more first sensors providing at least a measure of cell density and a measure of volume of a cell culture in the first chamber;
(b) one or more second bioreactors comprising:
(i) a second chamber; and
(ii) one or more second sensors providing at least a measure of culture conditions and a measure of volume of a cell culture in the one or more second chambers;
wherein the first chamber is in fluidic communication with the one or more second chambers;
(c) one or more reagent reservoirs comprising liquid reagents, wherein the reagent reservoirs are in fluidic communication with the first chamber and, optionally, with the one or more second chambers;
(d) one or more pumps configured to:
(i) move liquid reagents from at least one reagent reservoir to the first chamber;
(ii) move cell culture fluid from the first chamber to the one or more second chambers;
(iii) move cell culture fluid out of the one or more second chambers;
(iv) optionally move liquid reagents from at least one reagent reservoir to the one or more second chambers; and
(e) a control system configured to:
(i) use measures from the one or more sensors to control cell growth, e.g., cell density, of a culture of cells in the first chamber;
(ii) move cell culture fluid from the first chamber to the one or more second chambers; and
(iii) use measures from the one or more sensors to control culture conditions and, e.g., volume and/or cell growth, of a culture of cells in the one or more second chambers.
